(19) United States
(12) Patent Application Publication
(10) Pub. No.: US 2004/0137572 A1
(43) Pub. Date: Jul. 15, 2004

(54) Compositions and methods for generating conditional knockouts

(75) Inventors: Robert E. Finney, Shoreline, WA (US); Alan Lofquist, Kirkland, WA (US)

Correspondence Address: DONALD W. WYATT, PANGENEX, INC., 501 ELLIOTT AVENUE WEST, #400, SEATTLE, WA, 98119, US

(73) Assignee: PanGenex, Inc.

(21) Appl. No.: 10/741,083

(22) Filed: Dec. 20, 2003

Related U.S. Application Data
(63) Continuation of application No. 10/441,923, filed on May 19, 2003.
(60) Provisional application No. 60/425,032, filed on Nov. 8, 2002.
(60) Provisional application No. 60/382,069, filed on May 20, 2002.

(51) Int. Cl. C12P 21/02; C12N 5/06

(52) U.S. Cl. 435/69.1; 435/320.1; 435/325

(57) ABSTRACT

The present invention provides vectors and methods for the generation of conditional knockout and knockdown cells and animals. Vectors of the invention may be used to knockout or knockdown an endogenous gene and conditionally regulate the expression of an endogenous or ectopic gene. Accordingly, the invention provides vectors and methods useful for the identification of disease-associated genes, generating animal models of disease, and identifying drug candidates.

### CROSS-REFERENCES TO RELATED APPLICATIONS

This application is a continuation of U.S. patent application Ser. No. 10/441,923, filed May 19, 2003, which claims the benefit of U.S. provisional patent application Serial No. 60/425,032, filed Nov. 8, 2002. This patent application also claims the benefit of U.S. provisional patent application 60/382,069 filed May 20, 2002. All of these priority applications are herein incorporated by reference.

### BACKGROUND OF THE INVENTION

Many human diseases and disorders are currently without satisfactory medical treatments, due either to lack of efficacious drugs or due to unacceptable toxicities associated with current treatments. The inability to meet these medical demands is largely attributed to lack of validated targets (with acceptable efficacy and toxicity profiles) from which drug discovery and development can proceed. Because genes within the human genome and, accordingly, the products expressed thereby represent the majority of potential targets for drug discovery and development, substantially complete drafts of the entire genome sequences for humans and other species represent great opportunities for pharmaceutical companies to meet the vast numbers of unmet medical needs. However, simply knowing what genes are present in the human genome is not sufficient to determine which of the genes represent valid targets for drug discovery and development (e.g., what therapeutic indications are relevant for particular genes and which genes have acceptable toxicity profiles if drugs are developed for human use). Technologies that can interpret (identify) and test genes for efficacy (functions and therapeutic utilities) and toxicity profiles are therefore now in high demand.

Several approaches have emerged to assist with validation of genes (gene products) for drug discovery. Many of these are designed to correlate genes with human diseases and disorders. For example, DNA array technology is often used to measure levels of gene expression to associate particular genes with specific tissues, diseases, and disorders. Analyses of single nucleotide polymorphisms (SNPs) are used to associate mutations in human DNA sequences with specific diseases and disorders. An objective of proteomics is to associate proteins with one another and with signaling pathways that may be important for human diseases and disorders. Direct examination of predicted structures of gene products identified in the human genome and comparisons to gene products with known functions (either other human genes or non-human organisms) can also provide information as to which genes may have therapeutic value.

Other technologies strive to gain direct information about effects of gene product inactivation on human disease and disorder characteristics (e.g., to mimic the effects of a drug targeting that gene). These technologies include use of antisense RNAs, ribozymes, dsRNAi molecules, discovery of compound inhibitors of gene product activities, and gene knockouts. Antisense RNA, ribozyme, and dsRNAi technologies strive to target messenger RNA, an intermediate between the gene and the final gene product, which is usually a protein. Another means to inactivate gene products is through the use of chemical inhibitors. For this procedure, the biochemical function of the protein gene product is typically needed (e.g., a kinase, protease, transport protein, etc). With this information, high throughput screening assays are established, a chemical diversity library obtained, and selection of inhibitors of biochemical activity performed. Cells in culture or animals are then treated with the chemical inhibitors to determine effects of the inhibitor (and, therefore, presumably the gene product) on disease and disorder characteristics. Typically, the chemical entities used in the target validation stage are not highly active or specific for the gene product. Such reagents are usually obtained only after extensive pharmacological chemistry that is typically reserved for drug discovery programs.

Arguably, the most unambiguous means to demonstrate the functions and therapeutic utilities of genes is through direct genetic inactivation by gene knockout technologies. The strategy in cell culture involves the use of homologous recombination vectors to change (disrupt) the gene residing in the chromosome. Additional means to inactivate genes include gene trap technologies, which, like homologous recombination, inactivate the gene residing in the chromosome. Knockout animals may be produced from knockout cells produced in culture, for example.

The advantages of gene knockouts for determining the functions of genes are numerous. In particular, gene inactivation is specific for the targeted gene, and gene inactivation is complete (e.g., 100% inactivation if all copies of the gene are disrupted). There are no non-target associated effects, as seen with chemical inhibitors that typically not only inhibit the desired protein (usually not 100%) but also many unknown proteins residing in the cell. Yet, if gene inactivation leads to lack of proliferation or cytotoxicity in cultured cells, cells would not be recovered following gene inactivation. Similarly, if gene inactivation leads to embryonic lethality in animals, analysis of gene knockouts on functions, including efficacy and toxicity profiles of children and adults, is greatly impaired. In addition, by chronically inactivating genes, the cells or animals are allowed to adapt to loss of the gene product over time by changing expression or activities of other gene products to compensate. Moreover, the purpose of gene inactivation is to mimic the effects of a drug. Many (if not most) drugs are administered acutely, such that the gene product is inhibited for a short time. To model for the effects of acute administration more accurately, it is desirable to be able to regulate gene activation/inactivation.

To accommodate these issues, it is typically necessary to establish conditional expression systems. To accomplish this, a gene product is often expressed from a conditional promoter at an ectopic location in the chromosome. Gene inactivation by homologous recombination is then performed under permissive conditions (where expression is permitted from the conditional promoter) to inactivate the endogenous genes. Once the endogenous copies of the gene are inactivated, the effect of gene inactivation is observed by shifting the cells to non-permissive conditions (where the gene is no longer permitted to be expressed from the conditional promoter).

The use of conditional promoters to allow recovery of cells (animals) with inactivated genes for examination has many undesirable characteristics. Conditional systems must first be established for each desired gene. In particular, considerable optimization is required to ensure that expression is very low under non-permissive conditions, while at the same time ensuring adequate expression under permissive conditions. For cultured cells, expression from the ectopic (conditional) promoter is not under normal cellular controls. Hence, proteins normally expressed only in certain stages of the cell cycle or only in response to other gene products or activation signals are expressed constantly through use of the conditional promoter. In addition, the levels of expression are determined by the conditional promoter, and not by the natural, endogenous promoter. Typically, this means expression is abnormally high, which can lead to abnormal localization of the protein product in the cell and/or abnormal effects on natural or unnatural substrates in the cell. In addition, if abnormally expressed, the cells may have the ability to adapt to the abnormally expressed gene product by suppressing or enhancing expression of activity of other gene products in the cells.

In animal systems, additional undesirable effects are observed. In particular, the gene product expression from the conditional promoter is not only in tissues and cells where the protein would normally be found, but also in tissues and cells where the protein would not normally be found. In addition, regulation of expression and the level of expression would be abnormal. This again allows for abnormal cellular location and substrate utilization by the gene as well as allowing abnormal adaptation of the cells to the gene product.

In summary, there is a need to identify functions and therapeutic utility of genes within the human genome to meet medical needs. Although there are many methods to correlate gene expression, the presence of mutations in DNA, or the association of proteins with signaling pathways, few methods are readily available to evaluate what effects (efficacy and toxicity) would be expected should particular gene products be specifically inactivated by a drug product. Current methods are often non-specific, labor intensive, costly, and give ambiguous results. Even using methods such as gene knockouts that give unambiguous results for the functions of genes, the inability to mimic the effects of acute or chronic gene inactivation in cell culture or mature animals limits the utility of these systems. Together, the lack of methods to unambiguously associate efficacy and toxicity profiles to genes leads to the low quality of drug discovery targets and an ensuing low probability for clinical success of drug candidates. Thus, there is a need in the art for conditional gene knockout systems wherein expression of the targeted gene is primarily controlled by normal, endogenous regulatory elements under permissive conditions, and wherein expression of the gene may be regulated at different times, for different durations of time, and in different tissues, as desired. Relatedly, there is a need for less labor intensive and more routine methods for establishing and optimizing conditional expression systems.

### BRIEF SUMMARY OF THE INVENTION

The present invention uses either gene trapping technology or homologous recombination to conditionally regulate the expression of an endogenous gene by the integration of regulatable exogenous DNA into the genome of a cell. Accordingly, the invention provides vectors and related methods, as described below.

The invention provides vectors capable of introducing a regulatable gene expression inhibition element into a gene. These vectors include gene trap or insertion vectors, as well as homologous recombination or targeting vectors.

In one embodiment, the invention provides a vector containing a first polynucleotide sequence comprising a regulatable gene expression inhibition element and a marker cassette comprising in operable combination: (1) a first recombinase target site; (2) a second polynucleotide sequence encoding a marker; and (3) a second recombinase target site, wherein the first recombinase target site is located 5′ of the marker and the second recombinase site is located 3′ of the marker, and wherein said first polynucleotide sequence is located 5′ or 3′ of the cassette. In certain embodiments, the gene expression inhibition element is a transcription termination sequence, an mRNA disruption sequence, a transcription repressor target sequence, or a splice acceptor site.

In a related embodiment, the invention includes a vector containing a first polynucleotide sequence comprising a regulatable gene expression inhibition element, a second polynucleotide sequence encoding a marker, and an internal ribosome entry site, wherein said second polynucleotide sequence is located 3′ of said first polynucleotide sequence, and wherein said internal ribosome entry site is located 3′ of the second polynucleotide sequence.

In another embodiment, the invention includes a targeting vector containing a first polynucleotide sequence comprising a regulatable gene expression inhibition element, a marker cassette comprising in operable combination: (1) a first recombinase target site; (2) a second polynucleotide sequence encoding a marker; and (3) a second recombinase target site, and a first genomic sequence located 5′ or 3′ of the first polynucleotide sequence and the marker cassette. The first recombinase target site is located 5′ of the marker and the second recombinase site is located 3′ of the marker; the first polynucleotide sequence is located 5′ or 3′ of the marker cassette, the genomic sequence is located 5′ or 3′ of the first polynucleotide sequence and the marker cassette; and the genomic sequences correspond to polynucleotide sequences within a targeted gene.

In yet another embodiment, the invention includes a targeting vector containing a first polynucleotide sequence comprising a regulatable gene expression inhibition element; a marker cassette comprising in operable combination: (1) a first recombinase target site, (2) a second polynucleotide sequence encoding a marker, and (3) a second recombinase target site; a first genomic sequence located 5′ of the first polynucleotide sequence and the marker cassette; and a second genomic sequence located 3′ of the first polynucleotide sequence and the marker cassette. The first recombinase target site is located 5′ of the marker and the second recombinase site is located 3′ of the marker; the first polynucleotide sequence is located 5′ or 3′ of the marker cassette; the first and second genomic sequences are located 5′ and 3′ of the first polynucleotide sequence and the marker cassette, respectively; and the genomic sequences correspond to polynucleotide sequences within a targeted gene. In certain embodiments, the gene expression inhibition element is a transcription termination sequence, an mRNA disruption sequence, a transcription repressor target sequence, or a splice acceptor site. In one embodiment, the first and second genomic sequences comprise untranslated regions of the native gene. In another embodiment, the first and second genomic sequences comprise transcribed regions of the native gene located 5′ of a translation initiation site within the native gene. In yet another embodiment, the first and second genomic sequences comprise untranslated regions of the native gene located 3′ of a stop codon within the native gene. In one embodiment, the first and second genomic sequences comprise sequences within an intron of the native gene.

In specific embodiments of the vectors of the invention, the marker is a reporter, a positive selection marker, a negative selection marker, a positive switch marker, or a positive-negative selection marker. A vector may contain both a positive selection marker and a negative selection marker, or any other combination of markers. In one embodiment, a negative selection marker is located 5′ of the first genomic sequence or 3′ of the second genomic sequence. In one embodiment, the marker cassette further comprises a promoter sequence capable of driving expression of the marker.

In certain embodiments, vectors of the invention are a plasmid, a virus, a retrovirus, or a bacteriophage.

In certain embodiments, vectors of the invention may also include a transcription termination sequence located 5′ of the first genomic sequence, a polyA sequence located 3′ of the marker and 5′ of the second recombinase target site, and/or an IRES located 5′ of the marker and 3′ of the first recombinase target site.

In one embodiment, the gene expression inhibition element is capable of being regulated by a regulatory molecule. In specific embodiments, the regulatory molecule is a transcriptional repressor, a transcription terminator, an mRNA destabilizing molecule, an antisense RNA, a ribozyme, an siRNA, an shRNA, or a dsRNAi. In one embodiment, the gene expression inhibition element is capable of being regulated by the human immunodeficiency virus type I tat protein, or a variant thereof.

In other embodiments, the invention provides methods of disrupting the expression of a gene. In one embodiment, the invention provides a method of disrupting the expression of a specific gene within a eukaryotic cell, comprising introducing a targeting vector of the invention into the cell, wherein the first and second genomic sequences correspond to the specific gene.

In another embodiment, the invention provides a method of randomly disrupting the expression of a gene within a eukaryotic cell, comprising introducing a gene trap or insertion vector of the invention into the cell. In specific embodiments, the eukaryotic cell may be a mammalian cell, including a human cell, a murine cell, a rodent cell, or a primate cell. The mammalian cell may be a stem cell, such as an embryonic stem cell, for example. In certain embodiments of the invention, a vector of the invention is introduced in a cell by electroporation, transfection, microinjection, infection, gene gun, lipofection, or retrotransposition.

In another embodiment, the invention provides a method of generating a library of randomly mutated eukaryotic cells comprising introducing a vector of the invention into a multitude of eukaryotic cells to produce randomly mutated eukaryotic cells.

In a related embodiment, the invention includes a eukaryotic cell comprising within an endogenous gene an exogenous regulatable gene expression inhibition element and a marker cassette as described above, wherein the first recombinase target site is located 5′ of the marker and the second recombinase site is located 3′ of the marker, wherein said gene expression inhibition element is located 5′ or 3′ of the cassette, and wherein expression of said endogenous gene is disrupted.

In yet another related embodiment, the invention includes an animal comprising within an endogenous gene an exogenous regulatable gene expression inhibition element and a marker cassette as described above, wherein the first recombinase target site is located 5′ of the marker and the second recombinase site is located 3′ of the marker, wherein said first polynucleotide sequence is located 5′ or 3′ of the cassette, and wherein expression of said endogenous gene is disrupted.

In another embodiment, the invention provides a method of restoring expression of a disrupted gene within a cell or animal of the invention comprising delivering to the cell or animal a recombinase capable of excising the polynucleotide sequence encoding the marker, wherein excision of the polynucleotide sequence encoding the marker restores expression of the disrupted gene. In specific embodiments, the recombinase is cre or flp.

In another embodiment, the invention provides a eukaryotic cell or transgenic animal comprising, within an endogenous gene, an exogenous regulatable gene expression inhibition element, wherein expression of said endogenous gene is approximately normal.

In a related embodiment, the invention includes a eukaryotic cell or transgenic animal comprising, within an endogenous gene, an exogenous regulatable gene expression inhibition element, wherein expression of said endogenous gene is capable of being regulated by the regulatable gene expression inhibition element.

In certain embodiments, a cell or animal of the invention may also contain a regulatory molecule or a polynucleotide sequence encoding the regulatory molecule, wherein the regulatory molecule is capable of regulating expression of the endogenous gene via the regulatable gene expression inhibition element. In one embodiment, the regulatory molecule or polynucleotide sequence encoding the regulatory molecule is endogenous to the cell. In another embodiment, the regulatory molecule or polynucleotide sequence encoding the regulatory molecule is exogenous to the cell. In certain embodiment, the polynucleotide sequence encoding the regulatory molecule is present as a transgene. In one embodiment, the regulatory molecule is capable of being regulated, while in another embodiment, amounts of the regulatory molecule within the cell are capable of being regulated. In another embodiment, the activity of the regulatory molecule is capable of being regulated. In one embodiment, the regulatory molecule is expressed in a tissue-specific or temporally-restricted pattern.

In yet another embodiment of the invention, the invention provides a method of introducing a regulatable gene expression inhibition element into an endogenous gene in a cell, comprising introducing at least a portion of a vector of the invention into the gene and introducing a recombinase into the cell, wherein said recombinase excises the polynucleotide sequence encoding the marker.

In a related embodiment, the invention includes a method of regulating the expression of an endogenous gene, comprising introducing a regulatable gene expression inhibition element into an endogenous gene in a cell according to a method of the invention and introducing a regulatory molecule into the cell, wherein said regulatory molecule is capable of regulating said regulatable gene expression inhibition element and expression of the endogenous gene in the cell.

Another embodiment of the invention includes a method of regulating the expression of an endogenous gene by introducing a regulatable gene expression inhibition element into an endogenous gene in a cell according to a method of the invention and altering the activity of a regulatory molecule within the cell, wherein the regulatory molecule is capable of regulating the regulatable gene expression inhibition element and expression of the endogenous gene in the cell.

In a similar embodiment, the invention includes a method of producing a conditionally expressed gene within an animal by introducing a least a portion of a vector of the invention into an endogenous gene in an embryonic stem cell, introducing a recombinase into the cell, generating an animal produced from the cell, mating the animal with a second animal containing a transgene encoding a first regulatory molecule capable of regulating the regulatable gene expression inhibition element within the vector, and producing an offspring containing within its genome the vector and transgene, wherein the recombinase excises the marker from the portion of the vector; wherein the transgene is capable of being regulated by a second regulatory molecule; and wherein the first and second regulatory molecules are different molecules.

In another embodiment, the invention provides a method of producing a conditionally expressed gene within an animal by introducing a portion of a vector of the invention into an endogenous gene in an embryonic stem cell, introducing a recombinase into the cell, introducing a transgene capable of expressing a first regulatory molecule into the genome of the cell, and generating an animal from the cell, wherein the recombinase excises the marker from the portion of the vector; wherein the transgene is capable of being regulated by a second regulatory molecule, wherein the first and second regulatory molecules are different molecules. One or more of the steps may be performed in different orders.

In yet another related embodiment, the invention includes a method of generating a conditional knockout animal by homologous recombination by introducing a targeting vector of the invention into a multitude of ES cells, selecting an ES cell that underwent homologous recombination with the targeting vector, introducing a recombinase into the ES cell, generating an animal from the ES cell, mating the animal with an animal containing a transgene regulatably expressing a first regulatory molecule, and producing an offspring from the mating containing within its genome the regulatable transcription inhibition element of the targeting vector and the transgene, wherein the recombinase excises the marker; wherein the first regulatory molecule is capable of regulating the regulatable transcription inhibition element of the targeting vector; wherein expression of the transgene is capable of being regulated by a second regulatory molecule; and wherein the first and second regulatory molecules are not identical.

In one embodiment, the invention includes a method of regulating the expression of a gene in a cell by providing a second regulatory molecule to a cell containing: (1) an exogenous regulatable element within an endogenous gene and (2) a polynucleotide sequence encoding a first regulatory molecule capable of regulating the exogenous regulatable element, wherein the second regulatory molecule regulates expression of the first regulatory molecule, and wherein introduction of the second regulatory molecule alters expression levels of the endogenous gene.

In another embodiment, the invention includes a method of regulating the expression of a gene in a cell by providing a second regulatory molecule to a cell containing: (1) an exogenous regulatable element within an endogenous gene and (2) a first regulatory molecule capable of regulating the exogenous regulatable element, wherein the second regulatory molecule regulates the activity of the first regulatory molecule, and wherein introduction of the second regulatory molecule alters expression levels of the endogenous gene. In certain embodiments of the previous methods, the first regulatory molecule regulates transcription or mRNA stability. In specific embodiments, the first regulatory molecule is a transcription termination molecule, a transcription repressor, an mRNA disruption molecule, a ribozyme, an siRNA, a dsRNA, an shRNA, and an antisense RNA. In one embodiment, the first regulatory molecule is HIV-1 tat. In one embodiment, the second regulatory molecule is a transcription factor.

In another embodiment, the invention includes a conditional gene knockout system, comprising a recombinant vector of the invention, a cell comprising a transgene capable of expressing a regulatory molecule, wherein the regulatory molecule is capable of regulating the regulatable gene expression inhibition element of the recombinant vector; and a means for regulating the transgene or the regulatory molecule expressed therefrom. In specific embodiments, expression of the transgene is tissue- or developmental-stage specific or cell-cycle specific. In certain embodiments, expression of the transgene is regulated by a second regulatory molecule. In another embodiment, the activity of the regulatory molecule is regulated by a second regulatory molecule. In certain embodiments of the system, the second regulatory molecule is a transcriptional activator, a transcriptional repressor, a ligand, a receptor, an agonist, an antagonist, a binding partner, or an antibiotic. In one embodiment, the second regulatory molecule is tetR-VP16 or tetR^{mt}-VP16.

In another embodiment, the invention provides a method of producing a conditional knockout cell, comprising introducing into a multitude of cells a gene trap vector of the invention, selecting for cells wherein at least a portion of the gene trap vector integrated within an endogenous gene, introducing a recombinase into the cells, and selecting for a cell wherein the recombinase excised the marker from the integrated gene trap vector.

In yet another embodiment, the invention provides a method of producing a conditional knockout cell by homologous recombination, comprising introducing into a multitude of cells a targeting vector of the invention, selecting for cells that underwent homologous recombination with the targeting vector, introducing a recombinase into the cells, and selecting for a cell wherein the recombinase excised the selection marker.

In one embodiment, the invention provides a method of determining the function of a gene, comprising providing at least two knockout cells prepared according to a method of the invention, wherein the cells contain the same regulatable gene expression inhibitor element within the same gene, introducing a regulatory molecule to one of the cells, wherein said regulatory molecule alters the expression of the gene via the regulatable gene expression inhibitor element, and comparing a biological trait of the cell to that of a cell wherein a regulatory molecule is not introduced.

Another embodiment of the invention is a method of determining the function of a gene by providing a cell prepared according to a method of the invention, introducing a regulatory molecule to the cell; and comparing a biological trait of the cell before and after introduction of the regulatory molecule.

In a related embodiment, the invention provides a method of determining the function of a gene by providing at least two cells of the invention, wherein the cells contain the same regulatable gene expression inhibitor element within the same gene, introducing a regulatory molecule to one of the cells, wherein the regulatory molecule alters the expression of the gene via the regulatable gene expression inhibitor element; and comparing a biological trait of the cell to that of a cell wherein a regulatory molecule is not introduced.

Another embodiment of the invention includes a method of determining the function of a gene by providing a cell of the invention, introducing a regulatory molecule to the cell, and comparing a biological trait of the cell before and after introduction of the regulatory molecule.

In a related embodiment, the invention provides a method of determining the function of a gene by providing at least two animals prepared according to a method of the invention, wherein the animals contain the same regulatable gene expression inhibitor element within the same gene, introducing a regulatory molecule to an animal, wherein the regulatory molecule is capable of altering expression of the gene via the regulatable gene expression inhibitor element, and comparing a biological trait of the animal to that of an animal wherein a regulatory molecule is not introduced.

In another embodiment of the invention, the invention includes a method of determining the function of a gene by providing an animal prepared according to a method of the invention, introducing a regulatory molecule to the animal; and comparing a biological trait of the animal before and after introduction of the regulatory molecule.

Similarly, in another embodiment, the invention includes a method of determining the function of a gene by providing one or more animals of the invention, wherein the animals contain the same regulatable gene inhibitor element within the same gene, introducing a regulatory molecule to one of the animals, wherein the regulatory molecule alters the expression of the gene via the regulatable gene expression inhibitor element; and comparing a biological trait of the animal to that of an animal wherein a regulatory molecule is not introduced.

In one embodiment, the invention provides a method of determining the function of a gene by providing an animal of the invention, introducing a regulatory molecule to the animal; and comparing a biological trait of the animal before and after introduction of the regulatory molecule.

Another embodiment of the invention includes a method of identifying a gene with a specific function by providing a multitude of cells of the invention, introducing a regulatory molecule to the cells, wherein the regulatory molecule alters the expression of the gene in at least one cell, identifying a cell wherein a specific function is altered after introduction of the regulatory molecule, and identifying a disrupted gene within the cell with the altered function. In one embodiment, identification of the disrupted gene precedes the introduction of a regulatory molecule. In another, it follows.

Another embodiment of the invention includes a method of identifying a gene with a specific function by providing a multitude of animals of the invention, introducing a regulatory molecule to the animals, wherein the regulatory molecule alters the expression of the gene in at least one animal, identifying an animal wherein the specific function is altered after introduction of the regulatory molecule, and identifying a disrupted gene within the animal identified.

In another embodiment of the invention, the invention includes a method of verifying whether a gene is associated with a particular function by providing a cell of the invention, wherein the gene contains a regulatable gene expression inhibitor element, introducing a regulatory molecule to the cell, wherein the regulatory molecule is capable of altering expression of the gene, and examining a trait of the cell before and after introduction of the regulatory molecule, wherein the presence or absence of the trait indicates the gene is associated with the particular function.

In yet another embodiment, the invention provides a method of generating an animal model of a disease, comprising producing an animal of the invention, wherein the endogenous gene is associated with a disease.

In a related embodiment, the invention includes a method of generating an animal model of a disease by generating a knockout animal according to a method of the invention, wherein the animal contains a regulatable gene expression inhibitor element within a gene associated with a disease, and wherein treatment with a regulatory molecule causes traits associated with the disease.

Another embodiment of the invention provides a method of generating an animal model of a disease by generating a multitude of animals of the invention, introducing a regulatory molecule to the animals, and identifying an animal with a trait associated with the disease.

In another embodiment, the invention includes a method of generating an animal model of a disease comprising mating an animal of the invention with an animal model of a disease.

Another embodiment of the invention includes a method of identifying a compound capable of altering the expression or function of a gene, comprising contacting a cell of the invention with a regulatory molecule, wherein the regulatory molecule is capable of regulating the regulatable gene expression inhibition element, contacting the cell with a candidate compound, and comparing expression of a gene before and after contact with the compound.

In a related embodiment, the invention includes a method of identifying a compound capable of altering the expression or function of a gene, comprising: providing a cell of the invention, wherein the regulatable gene expression inhibitor element is located within the gene, contacting a cell with a regulatory molecule capable of altering expression of the gene, and comparing a biological trait in the cell after treatment with the regulatory compound with a biological trait in a cell before treatment, wherein the cells are also treated with a candidate compound.

In another embodiment, the invention provides a method of identifying a candidate compound capable of compensating for the loss of expression of a gene, comprising providing a cell of the invention, introducing a regulatory molecule to the cell, treating the cell with a candidate compound; and determining if treatment restored a biological trait associated with the cell that was altered after introduction of the regulatory molecule.

Another embodiment of the invention provides a method of producing a compound capable of altering expression of a gene by identifying a compound according to a method of the invention and purifying said compound.

A related embodiment of the invention provides a process for the manufacture of a compound capable of altering expression of a gene including identifying an inhibitor or enhancer of caspase-mediated apoptosis according to a method of the invention, derivitizing the compound, and optionally repeating at least one of the steps of identification or derivitization, to produce a compound capable of altering expression of a gene.

[summary of invention related to optimization of conditional expression system . . . to be completed when claims finalized]

[summary of invention related to preparation of conditional expression system . . . to be completed when claims finalized]

### DETAILED DESCRIPTION OF THE INVENTION

The current invention provides vectors and methods for conditionally regulating gene expression in cells and animals. The invention also provides conditional knockout cells and animals generated according to these methods. In addition, the invention provides methods of using these conditional knockout cells and animals to study gene function, to study diseases, and to screen and identify compounds that effect gene function, expression, and/or control disease onset or progression.

According to one embodiment of the invention, an endogenous gene is placed under conditional regulation by introducing a regulatable gene expression inhibition element within an untranslated region of the gene, so that expression of the gene is essentially normal in the absence of an active regulatory molecule that regulates gene expression through the regulatable gene expression inhibition element. Expression of the endogenous gene may be disrupted by introducing or activating a regulatory molecule that inhibits gene expression via the regulatable gene expression inhibition element located within the endogenous gene.

The invention encompasses a variety of components, including vectors capable of introducing a regulatable gene expression inhibitor element into a gene, genes and genomes comprising a regulatable gene expression inhibitor element, cells and animals containing an exogenous regulatable gene expression inhibitor element within their genome, libraries and arrays of such vectors and cells, methods and systems for producing knockout cells and animals of the invention, methods of regulating the expression of a gene via a regulatable gene expression inhibitor element inserted within the gene, and methods of regulating the expression or activity of regulatory molecules provided in the invention.

The invention also encompasses numerous applications and uses for these components, including methods of identifying the function of a gene, methods of identifying a gene associated with a particular function or trait, and generating animal models of disease. The invention also provides methods of screening compounds and identifying compounds that effect gene function or expression. Thus, the invention provides methods of identifying compounds for use as therapeutics to treat a variety of diseases and disorders.

The invention further provides compositions and methods for the conditional knockdown of a gene. In one aspect of this embodiment, the invention provides a cell comprising a conditional expression vector comprising a regulatable promoter, a polynucleotide sequence, and a knockdown reagent that targets the endogenous gene sequence. In certain embodiments, the polynucleotide sequence has at least 90% identity to an endogenous gene sequence of the cell, is a degenerate variant of an endogenous gene sequence of the cell, is an endogenous gene sequence comprising a plurality of base substitutions, or is a sequence that encodes a polypeptide having at least 90% identity to a polypeptide encoded by an endogenous gene sequence of the cell, and the polynucleotide sequence of step is expressed in the presence of the knockdown reagent. The invention also provides an animal comprising the cell.

In a related embodiment, the invention provides a conditional expression system that includes the following steps: (a) a conditional expression vector comprising a regulatable promoter and a polynucleotide sequence that has at least 90% identity to an endogenous gene sequence of the cell, is a degenerate variant of an endogenous gene sequence of the cell, is an endogenous gene sequence comprising a plurality of base substitutions, or is a sequence that encodes a polypeptide having at least 90% identity to a polypeptide encoded by an endogenous gene sequence of the cell, and (b) an expression vector comprising a polynucleotide sequence that expresses a knockdown reagent that targets the endogenous gene sequence of step (a), wherein the polynucleotide sequence of step (a) encodes a functional polypeptide which is expressed in the presence of the knockdown reagent of step (b) at levels at least 50% of the level of expression of the corresponding endogenous gene in the absence of the knockdown reagent of step (b). In various aspects of this embodiment, the knockdown reagent is an antisense polynucleotides, a ribozyme, or a dsRNA. The dsRNA may be a short interfering RNA (siRNA) or a short hairpin RNA (shRNA), for example.

In related embodiment of this conditional expression system, the conditional expression vector is regulated by tet.

Another embodiment of the invention includes a method of regulating the expression of a gene in a cell, comprising the steps of: (a) introducing into the cell a conditional expression vector comprising a regulatable promoter and a polynucleotide sequence that has at least 90% identity to an endogenous gene sequence of the cell, is a degenerate variant of an endogenous gene sequence of the cell, is an endogenous gene sequence comprising a plurality of base substitutions, or is a sequence that encodes a polypeptide having at least 90% identity to a polypeptide encoded by an endogenous gene sequence of the cell, and (b) introducing into the cell a knockdown reagent that targets the endogenous gene sequence of step (a), wherein the polynucleotide sequence of step (a) encodes a functional polypeptide which is expressed in the presence of the knockdown reagent of step (b) at levels at least 50% of the level of expression of the corresponding endogenous gene in the absence of the knockdown reagent of step (b). This method may further comprise the step of introducing into the cell an agent that regulates the regulatable promoter. In various embodiment, the knockdown reagent is stably expressed in the cell. In certain embodiments, the knockdown reagent is an antisense polynucleotide, a ribozyme, or a double-stranded RNA (dsRNA). The double-stranded RNA may be a short interfering RNA (siRNA) or short hairpin RNA (shRNA), for example.

Yet another embodiment of the invention provides a vector comprising an inducible promoter, a site-specific recombinase site, and a marker gene. In one embodiment, the vector further comprises a multiple cloning site sequence. In another embodiment, the vector comprises two site-specific recombinase sites. The site-specific recombinase sites may be located 5′ and 3′ of the multiple cloning site sequence and marker gene, respectively. The site-specific recombinase target site may be located 3′ of the multiple cloning site sequence. In one embodiment, the vector is a retrovirus. In another embodiment, the marker gene is secretory alkaline phosphatase.

A further embodiment of the invention includes a conditional expression system, comprising two vector; the first vector comprising an inducible promoter, a site-specific recombinase site, and a marker gene, and the second vector comprising a promoter and a polynucleotide sequence encoding a transcription regulator, wherein the transcription regulator regulates expression of the marker gene.

A related embodiment includes a method of conditionally regulating the expression of a gene of interest, comprising introducing the two vectors of the conditional expression system into a cell, selecting for a cell that conditionally expresses the marker gene, introducing a polynucleotide sequence comprising a gene of interest and site-specific recombinase sites into a selected cell, and selecting for a cell wherein site-specific recombination has occurred, such that the transcription regulator regulates expression of the gene of interest. In one embodiment of this method, the gene of interest replaces the marker gene via site-specific recombination. In another embodiment, the gene of interest is inserted 3′ of the marker gene. In yet another embodiment, the polynucleotide sequence of step (d) further comprises an IRES 5′ of the gene of interest.

Another aspect of the invention includes a conditional expression system, comprising a vector comprising an inducible promoter sequence, a site-specific recombinase site, and a gene of interest; and a vector comprising a promoter and a polynucleotide sequence encoding a transcription regulator, wherein the transcription regulator regulate expression of the marker gene.

A related embodiment includes a method of conditionally regulating the expression of a gene of interest, comprising knocking out an endogenous gene of interest; and conditionally regulating the expression of the gene of interest according to a method of the invention. In one embodiment, the knockdown reagent used in the conditional regulation method is selected from the group consisting of: antisense polynucleotides, ribozymes, and dsRNA. The dsRNA is a siRNA or a shRNA.

In other embodiments, the invention includes eukaryotic cells comprising conditional expression systems of the invention. The invention further comprises libraries and arrays of these cell, wherein each cell comprises different genes of interest. In one embodiment, the array comprises multiple groups of vessels, of which at least two of said vessels each contains a cell (i) comprising the conditional expression system of claim 122 and (ii) arranged is said array in a predetermined fashion.

In another embodiment, the invention includes a transgenic animal comprising a cell comprising a conditional expression system of the invention. In various embodiments, the animal is a mammal, such as a mouse.

Another embodiment of the invention provides a method of identifying a compound that inhibits or enhances the activity of a gene product, comprising: (a)conditionally regulating a gene of interest in a cell according to a method of the invention; (b) contacting the cell of step (a) with a candidate compound; and (c) comparing a biological trait of the cell before and after step (b).

In a related embodiment, the invention provides a method of identifying a compound capable of compensating for the loss of expression of a gene, comprising (a) providing a cell comprising a conditional expression system of the invention; (b) introducing to the cell a compound that reduces expression of the conditionally regulated gene; contacting the cell of step (b) with a candidate compound; and (d) determining if contacting according to step (c) restored a biological trait associated with loss of expression of the conditionally regulated gene.

The invention includes numerous embodiments, as the invention may be successfully practiced in many different forms. Thus, the invention contemplates all types of vectors, regulatable gene expression inhibitor elements, regulatory molecules, means of regulating the expression or activity of genes and regulatory molecules, genomic insertion sites, etc., that may be utilized to practice the disclosed invention. The invention includes all suitable forms of each element and component of the invention, and every possible combination thereof. The invention includes both compositions and methods for both gene knockout and gene knockdown, as well as related methods of preparing and optimizing conditional expression systems. Specific embodiments of the invention are described in detail below.

A. Vectors

The invention includes all vectors known in the art that may be used to transfer an exogenous DNA sequence into the genome of a cell, including both insertion and replacement vectors. Vectors of the invention may be plasmids, viruses, or retroviruses, and the like, for example. Specific embodiments of the invention include gene trap vectors and targeting vectors. Generally, gene trap vectors are designed to randomly integrate within genes, while targeting vectors are designed to undergo homologous recombination with a specific genomic sequence. Methods of designing and constructing vectors, and methods of introducing vector sequences into a genome are well known in the art and are described, for example, in GENE TARGETING: A PRACTICAL APPROACH, 2nd ed. (2000), Joyner, A. L., ed., Oxford University Press, New York; GENE TARGETING PROTOCOLS (METHODS IN MOLECULAR BIOLOGY, VOL. 133), (2000), Kmiec, E. B. and Gruenert, D. C., eds., Humana Press; and Torres, R. M. et al., LABORATORY PROTOCOLS FOR CONDITIONAL GENE TARGETING (1997), Oxford University Press, Oxford; and references cited within, all of which are incorporated by reference. Specific vectors are also described in U.S. Pat. No. 5,364,783, No. 5,464,764 (positive-negative selection), No. 5,487,992, No. 5,627,059, No. 5,631,153, No. 5,719,055 (transposons), U.S. Pat. No. 5,830,698, No. 5,998,144, No. 6,280,937, No. 6,284,541, Nos. 6,139,833, 6,303,327, No. 6,319,692, No. 6,329,200, and No. 6,080,576, and references and patents cited therein. Generally, vectors of the invention may be constructed, propagated, isolated, and examined using routine molecular biology techniques such as restriction enzyme digestion, polymerase chain reaction, ligation, transformation, and southern blotting, according to procedures well known in the art and described in CURRENT PROTOCOLS IN MOLECULAR BIOLOGY, (2001), Ausubel et al. (eds.), John Wiley & Sons, New York and Sambrook, et al., MOLECULAR CLONING: A LABORATORY MANUAL, (2001), Cold Spring Harbor Laboratory Press, Cold Spring Harbor, N.Y., and U.S. Pat. No. 5,789,215, for example.

Vectors of the invention share several common features, including a regulatable gene expression inhibitor element (defined later) and a marker cassette comprising a marker flanked by two recombinase target sites. The marker cassette is provided in operable combination, wherein the recombinase target sites are positioned in an orientation and with spacing that permits the excision of intervening polynucleotide sequences, including those encoding the marker, in the presence of a recombinase that recognizes the recombinase target sites. In one preferred embodiment, the vectors have the following components: a regulatable gene expression inhibitor element, a first recombinase target site, a marker, and a second recombinase target site. In one embodiment, these elements are present in the described order (5′ to 3′) within the vector. Alternatively, the regulatable gene expression inhibitor element may reside 3′ to the most 3′ recombinase target site. The components are aligned so that introduction of a recombinase causes excision of sequence between the recombinase target sites, but leaves the regulatable gene expression inhibitor element intact within the gene. In certain embodiments, introduction of vector or marker sequences into the genome of a cell diminishes or alters expression of the disrupted gene. In certain embodiments, expression of a disrupted gene is substantially normal upon introduction of a recombinase.

In certain embodiments, vectors of the invention include a transcription termination sequence to the 3′ of the marker. In some embodiments, this transcription termination sequence is a polyadenylation (polyA) site. Generally, the polyA site is located 5′ of the most 3′ recombinase target site. However, it may also be located 3′ of the most 3′ recombinase target site, so long as the presence of the transcription termination sequence does not itself significantly alter the normal levels of expression of the disrupted gene or its encoded polypeptide as compared to normal levels, e.g., if the transcription termination sequence is inserted in the 3′ untranslated region of the gene. In preferred embodiments, the introduction of the transcription termination sequence into the disrupted endogenous gene will result in the expression of an mRNA transcript with a 3′ end determined by the transcription termination sequence. Thus, when the vector of the invention inserts into the 5′ untranslated region or an intron or exon of an endogenous gene, transcription will not proceed through the region of the disrupted gene located 3′ of the insertion site. PolyA sites include, for example, the SV40 polyA site, the phosphoglycerate kinase polyA site, and the bovine growth hormone polyA site, described in the Invitrogen 1996 catalog and Joyner, supra. Transcription termination sequences are widely known and available to those of skill in the art.

In certain embodiments, vectors of the invention include an internal ribosome entry site (IRES) sequence to the 5′ of the marker. The IRES sequence is generally located 3′ of the most 5′ recombinase target site. However, in certain embodiments, the IRES sequence can be located 5′ of the most 5′ recombinase site, so long as its presence does not substantially alter the level of expression of the disrupted gene or its encoded polypeptide compared to normal levels. Normal levels refer to levels expressed in the same cell from the endogenous, non-disrupted gene. Thus, normal levels include a range of expression levels equivalent to levels observed in the same cell. It is understood that there is natural variation in expression levels depending on a variety of factors, including, e.g. the length of time or number of passages that cells have been cultured, the source of the cell, and cell culture conditions. IRES sequences of the invention include, for example, those from encephalomyocarditis virus, poliovirus, piconaviruses, picona-related viruses, and hepatitis A and C. Examples of suitable IRES sequences are disclosed in European patent application 585983 and PCT applications WO/9611211, WO/9601324, and WO/9424301. In another embodiment, the IRES is from the 5′ leader sequence of the mRNA of the homeodomain protein Gtx, as is described in detail in Chappel, S. A. et al., Proc. Natl. Acad. Sci USA 97:1536-1541 (2000); Owens, G. C. et al., Proc. Natl. Acad. Sci. USA 98:1471-1476 (2001); and Hu, M. C.-Y. et al, Proc. Natl. Acad. Sci USA 96:1339-1344 (1999), all of which are incorporated by reference in their entirety.

In another embodiment of the invention, vectors comprise a regulatable gene expression inhibitor element, a marker gene located 3′ of the gene expression inhibitor element, and an IRES located 3′ of the marker gene. This embodiment may optionally include one or more recombinase binding sites. The presence of recombinase sites is optional in this vector, since removal of the marker may not be necessary, depending on where the vector inserts within the disrupted endogenous gene. For example, if the vector integrates into the 5′ untranslated region of a gene, the endogenous promoter will drive expression of a chimeric mRNA that encodes the marker and the endogenous gene. The presence of the IRES 5′ of the coding region of the endogenous gene facilitates loading of ribosomes and expression of the polypeptide encoded by the endogenous gene. Hence, both the marker and the endogenous gene would be under control of the regulated expression inhibitor sequence, according to this embodiment of the invention.

1. Gene Trap Vectors

Gene trap vectors of the invention include all vectors capable of inserting at least a portion of their polynucleotide sequence into a gene. Gene trap vectors are described, for example, in U.S. Pat. No. 6,218,123, No. 6,207,371, No. 6,139,833, and No. 6,080,576, and references cited within, all of which are hereby incorporated by reference in their entirety. Examples of gene trap vectors contemplated by the invention include promoter trap vectors, exon trap vectors, and 3′ (polyA) trap vectors. In addition, gene trap vectors contemplated by the invention include secretion trap vectors and conventional gene trap vectors designed for insertion into an intron. Preferably, a gene trap vector of the invention inserts within an untranslated region of a gene. This untranslated region may be located, e.g., to the 5′ or 3′ of coding regions of the gene, or it may reside within an intron or untranslated exon of the gene. Preferred examples of gene trap vectors of the invention include those comprising selectable marker genes that are not expressed unless particular properties or factors are included within or provided by endogenous cellular sequences flanking the vector after integration. Such factors include, not are not limited to, polyadenylation (polyA) sequences, active promoters, and splice donor and acceptor sites. For example, since the vector does not contain a promoter driving expression of the marker, the marker will not be expressed unless integrated into a transcriptionally active gene.

In one embodiment, gene trap vectors of the invention comprise a first polynucleotide sequence comprising a regulatable gene expression inhibition element, and a marker cassette comprising, in operable combination, a marker flanked by two recombinase target sites. The marker is preferably a selectable marker that allows the identification and selection of cells wherein vector sequences integrated within a gene. In certain embodiments, a gene trap vector of the invention may also contain a reporter, preferably between the recombinase sites of the marker cassette, useful for examining the expression patterns of the endogenous gene. Useful reporters include any detectable gene or any gene expressing a detectable gene product. Generally, reporter genes are not endogenous to the cell. Reporters are widely known in the art, as are methods of assaying reporter expression or enzymatic activity. Examples of reporters include, but are not limited to, alkaline phosphatase, β-galactosidase, fluorescent protein, and luciferase genes. Alternatively, a gene trap vector may contain a reporter/selection fusion cassette, such as those described in Joyner, supra. One example of a reporter/selection fusion cassette that may be used according to the invention is βgeo, a fusion between lacZ and neomycin resistance genes. See, for example, Friedrich, G. and Soriano, P., GENES DEV. 5:1513 (1991) and Schuster-Gossler, K. et al., TRANSGENE 1:281 (1994).

In certain embodiments, gene trap vectors are designed to facilitate the identification of cells wherein vector sequences have inserted into a region of an exon located upstream (5′) of a translation initiator sequence within the gene. In such embodiments, the gene trap vectors of the invention preferably do not contain splice acceptor or donor sites that allow fusion of the marker sequence with translated sequences of the gene and subsequent expression of a fusion protein that includes the marker. Marker expression is driven by endogenous promoter and regulatory sequences within the disrupted gene. Subsequent translation utilizes a translation initiator sequence within the marker and does not require splicing with endogenous gene sequences. Preferably, the marker is not expressed as a fusion protein with sequences from the endogenous gene. Typically, the marker will include a stop codon at the 3′ end of its coding sequence that terminates mRNA translation. In addition, in certain embodiments, a transcription termination sequence, such as a polyA sequence, is included within the marker cassette, following the sequence encoding the marker.

In other embodiments, the vector may include a promoter 5′ of the marker. Marker selection is, therefore, not necessarily dependent upon expression of the disrupted gene. Also, the marker may be expressed as its own polypeptide, rather than as a fusion protein containing sequences expressed from the disrupted gene. In preferred embodiments, the vector will also contain a polyA sequence 3′ of the marker. The invention contemplates the use of a variety of exogenous promoters to drive marker expression. In preferred embodiments, promoters will drive constitutive expression of sufficient levels of marker to facilitate detection or selection of cells that have integrated vector sequences. Examples of suitable promoters include human β-actin, phosphoglycerate kinase (PGK), and herpes simplex virus thymidine kinase (HSV-TK).

In certain related embodiments, 3′ gene trap vectors are designed to facilitate the identification of cells wherein vector sequence has inserted 3′ of the disrupted gene's coding sequences and 5′ of the gene's polyA sequence. In certain embodiments, these vectors are polyA trap vectors that include a promoter to drive expression of the marker, yet do not include a polyA sequence downstream of the marker. Thus, the marker can be transcribed independently of the disrupted gene, but it requires capture of the disrupted gene's endogenous polyA to produce a stable mRNA and permit identification.

In other embodiments, intron trap vectors are designed to facilitate the identification of cells wherein vector sequence has inserted into an intron of a gene. To facilitate expression of a fusion protein that includes the marker, such vectors may contain splice-acceptor and splice-donor sequences 5′ and 3′ of the marker, respectively. Splice donor and splice acceptor sequences are short consensus sequences at the exon/intron and intron/exon boundaries (splice-donor and splice-acceptor, respectively; as described in Joyner, supra). These vectors may also include a splice branch site located approximately 30 base pairs 5′ to the splice acceptor sequence (reviewed in Sharp, P. A., (1994), Cell, 77, 805). Any functional splice acceptor sequence may be used according to the invention, including, for example, mouse En-2, c-fos, and adenovirus major late gene splice acceptor sequences. In one embodiment, an intron trap vector according to the invention includes a splice acceptor sequence 5′ of the marker gene. The presence of the splice acceptor mediates splicing of upstream exon sequence to the marker gene and expression of a fusion protein comprising the polypeptide expressed by the marker gene. Fusion protein expression is driven by the endogenous promoter-associated with the disrupted gene.

2. Targeting Vectors

Targeting vectors of the invention include all vectors capable of undergoing homologous recombination with an endogenous gene, and, in certain embodiments, targeting vectors are replacement vectors. Targeting vectors include all those used in methods of positive selection, negative selection, positive-negative selection, and positive switch selection, in addition to any other targeting vector. Targeting vectors employing positive, negative, and positive-negative selection are well known in the art and representative examples are described in Joyner, A. L., GENE TARGETING: A PRACTICAL APPROACH, 2nd ed. (2000) and references cited therein, in addition to U.S. patents cited above. Vectors employing positive switch selection methods are described in U.S. patent application Ser. No. 10/028,970, filed Dec. 28, 2001, which is hereby incorporated in its entirety. Essentially, positive switch selection methods involve replacing an original selection marker sequence of a gene trap construct with a reporter sequence and/or a new selection marker sequence.

Targeting vectors of the invention comprise a first polynucleotide sequence comprising a regulatable gene expression inhibition element, and a marker cassette comprising, in operable combination, a marker flanked by two recombinase target sites. In addition, targeting vectors of the invention include one or more genomic sequences. In one embodiment, a targeting vector comprises two genomic sequences, which together are capable of directing integration of vector sequence into a corresponding gene. In another embodiment, a targeting vector contains only a single genomic sequence, which individually is capable of directing the integration of vector sequence into a corresponding gene via integration/excision methods. In certain embodiments, integration/excision methods are mediated via an integron vector, for example, as described in Ply, M. C. et al., CLIN. CHEM. LAB. MED, 38(6):483-7 (2000); Gravel, A. et al., NUCLEIC ACIDS RESEARCH 26:4347-4355 (1998); Shimuzu-Kadota, M. J. BIOTECHNOL. 89:73-9 (2001); Murphy, S. J., HUM. GENE THER. 13:745-60, and references cited within, all of which are incorporated by reference in their entirety.

Genomic sequences are polynucleotide sequences having the same or substantially the same sequence as a region of the genome of a target cell. Generally, a genomic sequence of the invention is a polynucleotide sequence corresponding to a region of a gene targeted for homologous recombination. This gene may also be referred to as a native gene, thereby indicating that it is an unmodified or wild-type gene. In one embodiment, the two genomic sequences of a targeting vector of the invention are located adjacent to each other within the corresponding gene within the target cell genome. However, in certain embodiments, the two genomic sequences may overlap each other in the native gene, or there may be additional polynucleotide sequence between the corresponding regions of the native gene. In certain embodiments, genomic sequences are selected such that the coding region of the targeted gene is not altered by homologous recombination with the targeting vector, so the polypeptide expressed by the gene following recombination and introduction of a recombinase is identical to the polypeptide expressed from the native gene. In addition, regulatory elements capable of regulating expression of the native gene are preferably not disrupted following homologous recombination with a targeting vector of the invention. In other embodiments, homologous recombination may result in alterations in target gene sequence, such as nucleotide deletion, insertion, or substitution, for example. Preferably, these alterations do not substantially alter or disrupt the function or expression of the gene product. In other embodiments, however, it may be desirable to introduce a mutation that alters the sequence, function, or expression of the gene product. For example, such alterations may be useful for gene or protein structure-function studies or for studying the effects of altering protein expression. Alterations produced in certain embodiments of the invention include amino acid substitutions, insertion, and deletions, for example.

In certain embodiments, the genomic sequences correspond to an untranslated region of a target gene. However, translated regions of a target gene may be included within genomic sequences. If a translated region is included, it is usually only included within one genomic sequence of a targeting vector, so polynucleotide sequences of the targeting vector that do not correspond to sequences within the native gene are inserted outside of the translated region of the native gene. For example, in certain embodiments of the invention, the 5′ genomic sequence of the vector may contain a translated region corresponding to the most 3′ translated region of the native gene. In another embodiment, the 3′ genomic sequence may contain a translated region corresponding to the most 5′ translated region of the native gene. Alternatively, both genomic sequences may include exon sequence, such as where each genomic sequence contains an exon sequence adjacent to either end of an intron, respectively.

In one embodiment, homologous recombination vectors of the invention may be used to disrupt target gene expression by targeting insertion of vector DNA into into translated regions of a gene. For example, a vector may be constructed that inserts vector sequence into a transcribed region located 5′ of a translation start site of a gene. In other embodiments, a vector may be constructed to insert vector sequence into a transcribed region located 3′ of a translation stop codon. Alternatively, a vector may insert vector sequence into an intron of a gene. Design and construction of homologous recombination vectors is well known to those of skill in the art, and the site of homologous recombination within an endogenous gene may be directed by the choice of genomic sequences, for example.

Homologous recombination vectors of the invention generally contain markers capable of being used to identify and select for insertion of vector sequence into a gene. If using a positive-negative system, a negative selection marker is placed either 5′ to the 5′ genomic sequence or 3′ to the 3′ genomic sequence. In certain embodiments, the vector contains a promoter that drives expression of the marker gene. Examples of suitable promoters include, but are not limited to, phosphoglycerate kinase I (PGK) RNA polymerase II, metallothionein, β-actin, SV40, immunoglobulin, human cytomegalovirus and thymidine kinase promoters, such as the synthetic mutant polyoma enhanced herpes simplex virus thymidine kinase, MC1. In certain embodiments, the marker does not have its own promoter, and the endogenous gene promoter drives marker expression. If the targeted gene is expressed in the cell, gene sequences may drive expression of the marker inserted by homologous recombination. However, where a target gene is not expressed or expressed at low levels in a target cell, a promoter is typically included 5′ to the marker, but 3′ to the 5′ recombinase target site.

3. Regulatable Gene Expression Inhibitor Elements

A regulatable gene expression inhibitor element of the invention is any sequence that may be regulated to alter expression of an associated gene. A regulatable gene expression inhibitor element may be capable of regulating gene expression at any step, including transcription initiation, transcription elongation, transcription termination, mRNA stability, RNA splicing, and translation, for example. Regulatory elements and molecules that control gene expression are well known in the art. Regulatable gene expression inhibitor elements are generally targets for regulation by a corresponding regulatory molecule or compound. For example, regulatable gene expression inhibitor elements include. transcription termination sequences, transcription factor binding sites, ribozyme target sites, splice acceptor sites, dsRNAi target sequences, short interfering RNA (siRNA) target sequences, short hairpin RNA (shRNA) target sequences, and antisense RNA targets. Regulatable gene expression inhibitor elements of the invention may mediate a reduction in expression of an associated gene in the presence of a corresponding regulatory molecule or compound. Alternatively, gene expression inhibitor elements of the invention mediate a reduction in expression of an associated gene upon removal of a regulatory compound.

Regulatory molecules and compounds include any molecule or compound capable of regulating gene expression via the regulatable gene expression inhibitor element, either directly or indirectly. In certain embodiments, a regulatory molecule or compound binds in a sequence-specific manner to a regulatable gene expression inhibitor element. In other embodiments, the regulatory molecule acts indirectly. For example, a regulatory molecule may be a binding partner for a molecule that interacts with the regulatable gene expression inhibitor element, or a regulatory molecule may promote the release of an inhibitory molecule from a molecule that binds a regulatable gene expression inhibitor element. A regulatory molecule may also, e.g., act by activating a second molecule that acts on the regulatable gene expression inhibitor element, or by altering subcellular localization of a molecule that acts directly on the regulatable gene expression inhibitor element.

Thus, in certain embodiments, the regulatory molecule or compound is an environmental inducing agent. Appropriate environmental inducing agents include, for example, exposure to heat, various steroidal compounds, divalent cations (including Cu⁺² and Zn⁺²), galactose, tetracycline, isopropyl β-D thiogalactosidase (IPTG), as well as other naturally occurring and synthesized agents. Again, it is important to note that in certain embodiments of the invention, an environmental inducing agent can correspond to the removal of any of the above listed agents which are otherwise continuously supplied in the uninduced state (see, e.g., tTA based system described below).

In certain embodiments, transcription termination sequences are used to regulate the regulatable polynucleotide of the invention. A transcription termination sequence is any polynucleotide sequence whose presence within a gene disrupts or terminates transcription of the gene. Transcription termination sequences include, for example, polyA sequences and the trans-acting responsive (TAR) element present just 3′ to the start site of transcription in the human immunodeficiency virus LTR. Binding of the viral protein tat and cellular proteins to the TAR allows full-length transcription of the retroviral genome. The presence of the TAR within the HIV LTR promoter essentially renders the promoter inactive in the absence of the tat gene product, which is expressed upon viral infection. A variety of elements and molecules regulating transcription termination are well known in the art and have been described, for example, in Zhao, J. et al., FORMATION OF MRNA 3′ ENDS IN EUKARYOTES: MECHANISM, REGULATION, AND INTERRELATIONSHIPS WITH OTHER STEPS IN MRNA SYNTHESIS, (1999), Microbiology and Molecular Biology Reviews, 63, 405-445. It should be understood that any regulatable transcription termination sequence may be used according to the invention.

In other embodiments, transcription repressors and corresponding binding sites may be used to regulate gene expression according to the invention, as well as silencers. Preferably, transcription repressors used in the invention are not endogenously expressed in the cell or animal containing a vector of the invention, or they do not alter the expression of non-disrupted endogenous genes when activated or introduced into a cell. However, endogenous transcription repressors and target sites may be used according to the invention. All that is required is that the addition or activation of the transcription repressor causes a reduction in expression of the disrupted gene as compared to normal levels of expression of the gene or encoded polypeptide.

Suitable transcription inhibitors include all those listed in the Transcription Regulatory Genes Database, described in Nucleic Acids Res., 28 (1) 298-301 (1999), as well as any other transcription repressor. Sequence-specific transcription repressors used according to the invention include those from prokaryotes and eukaryotes, including bacteria, yeast, Drosophila and mammals, for example. Transcription repressor proteins may contain a variety of structural elements and motifs and include, for example, helix-loop-helix, leucine zipper, zinc finger, bromodomain, homeodomain, polycomb, ets family, and nuclear hormone receptor proteins. The use of transcription repressor systems to regulate gene expression within the mouse is described in Lewandowski, M., (2001), CONDITIONAL CONTROL OF GENE EXPRESSION IN THE MOUSE, Nat. Rev. Genet., 10: 743-755. Indeed, virtually any site-specific DNA binding protein can be adapted for use as a repressor in the present invention by fusing a domain capable of recognizing the regulatable gene expression inhibitor sequence to a transcription repression domain. Accordingly, virtually any DNA binding protein target site may be a regulatable gene expression inhibitor sequence of the invention. A variety of repression domains are known in the art, including for example, the N-terminus of the Mad family of basic-helix-loop-helix proteins, which recruits Sin3 and histone deacetylases, and the region of the yeast alpha 2 repressor that recruits the Ssn6-Tup1 complex in yeast. Repression domains of any species may be used, so long as they are capable of mediating repression within a cell containing a disrupted gene according to the invention. Without wishing to be bound to a particular theory, repression may be mediated by deacetylation or methylation, for example.

Transcription repressor elements have been identified in the promoter region of a variety of genes, including the collagen II gene, for example. Repressor elements have also been shown to regulate transcription in the carbamyl phosphate synthetase gene (Goping et al., NUCLEIC ACID RESEARCH 23 (10):1717-1721, 1995). Negative regulatory regions have been identified in the promoter region of the choline acetyltransferase gene, the albumin promoter (Hu et al., J. Cell Growth Differ. 3(9):577-588, 1992), phosphoglycerate kinase (PGK-2) gene promoter (Misuno et al., Gene 119(2):293-297, 1992), and in the 6-phosphofructo-2-kinase/fructose-2, 6-bisphosphatase gene, in which the negative regulatory element inhibits transcription in non-hepatic cell lines (Lemaigre et al., Mol. Cell Biol. 11(2):1099-1106). Furthermore, the negative regulatory element Tse-1 has been identified in a number of liver specific genes, including tyrosine aminotransferase (TAT). TAT gene expression is liver specific and inducible by both glucocorticoids and the cAMP signaling pathway. The cAMP response element (CRE) has been shown to be the target for repression by Tse-1 and hepatocyte-specific elements (Boshart et al., Cell 61(5):905-916, 1990). Accordingly, it is clear that a variety of such elements are known or are readily identified.

However, it must also be understood that a transcription repression element according to the invention does not necessarily function as a repression element or mediate transcription repression in its ordinary or native context. The only requirement for a transcription repression element of the invention is that it is capable of mediating transcription repression, e.g. when bound by a transcription repressor. Since a transcription repressor may be engineered to contain a repression domain fused to any sequence-specific DNA binding domain, a transcription repression element of the invention may, in fact, function as an enhancer or activator of transcription in a native context. Thus, an important characteristic of a transcription repression element is its ability to bind a sequence-specific transcription repressor.

In other embodiments, ribozymes are used to regulate gene expression. A ribozyme is an RNA molecule that specifically cleaves RNA substrates, such as mRNA, resulting in specific inhibition or interference with cellular gene expression. Generally, a ribozyme is an RNA that has both a catalytic domain and a sequence homologous to a particular mRNA. The ribozyme functions by associating with the mRNA (through the homologous domain of the ribozyme) and then cleaving (degrading) the message (using the catalytic domain). Ribozymes are described in more detail infra. Ribozymes can be targeted to any RNA transcript and can catalytically cleave such transcripts (see, e.g., U.S. Pat. No. 5,272,262; No. 5,144,019; and Nos. 5,168,053, 5,180,818, 5,116,742 and 5,093,246 to Cech et al.). Methods of designing and using ribozymes are known in the art, and are described, for example, in the aforementioned patents, as well as U.S. Pat. No. 5,334,711, No. 5,225,337, No. 5,625,047, No. 5,631,359, No. 6,022,962, and references cited within.

In yet another embodiment, antisense molecules are used to regulate gene expression. Antisense molecules are oligonucleotides that bind in a sequence-specific manner to nucleic acids, such as mRNA or DNA. Antisense RNA technology involves expressing or introducing an RNA molecule (or derivative) that is homologous to sequences found in a particular mRNA into a cell. By associating with the mRNA, the antisense RNA inhibits use of the mRNA for production of the protein product of the gene. (see, e.g., U.S. Pat. No. 5,168,053, U.S. Pat. No. 5,190,931, U.S. Pat. No. 5,135,917; U.S. Pat. No. 5,087,617, and Clusel et al. (1993) NUCL. ACIDS RES. 21:3405-3411, which describes dumbbell antisense oligonucleotides), all of which are hereby incorporated by reference in their entireties. Without wishing to be bound by a particular theory, antisense technology can be used to control gene expression through interference with binding of polymerases, transcription factors or other regulatory molecules (see Gee et al., In Huber and Carr, MOLECULAR AND IMMUNOLOGIC APPROACHES, Futura Publishing Co. (Mt. Kisco, N.Y.; 1994)). Alternatively, an antisense molecule may be designed to hybridize with a control region of a gene (e.g., promoter, enhancer or transcription initiation site), and block transcription of the gene; or to block translation by inhibiting binding of a transcript to ribosomes. For example, the desirable properties, lengths and other characteristics of such oligonucleotides are well known. Antisense oligonucleotides are typically designed to resist degradation by endogenous nucleolytic enzymes by using such linkages as: phosphorothioate, methylphosphonate, sulfone, sulfate, ketyl, phosphorodithioate, phosphoramidate, phosphate esters, and other such linkages (see, e.g., Agrwal et al., TETREHEDRON LETT. 28:3539-3542 (1987); Miller et al., J. AM. CHEM. Soc. 93:6657-6665 (1971); Stec et al., TETREHEDRON LETT. 26:2191-2194 (1985); Moody et al., NUCL. ACIDS RES. 12:4769-4782 (1989); Letsinger et al., TETRAHEDRON 40:137-143 (1984); Eckstein, ANNU. REV. BIOCHEM. 54:367-402 (1985); Eckstein, TRENDS BIOL. SCI. 14:97-100 (1989); Stein In: OLIGODEOXYNUCLEOTIDES. ANTISENSE INHIBITORS OF GENE EXPRESSION, pp. 97-117, Cohen, Ed, Macmillan Press, (London, (1989)); Jager et al., BIOCHEMISTRY 27:7237-7246 (1988)). Methods of designing and producing antisense molecules to disrupt expression through a particular sequence element are well known in the art and are described in further detail infra.

The procedure of double-stranded RNA interference (RNAi) may also be used to specifically inhibit expression of an associated gene. Briefly, the presence of double-stranded RNA dominantly silences gene expression in a sequence-specific manner by causing the corresponding endogenous RNA to be degraded. Although first discovered in lower organisms such as the nematode and Drosophila, for example, dsRNAi has been demonstrated to work in mammalian cells (Wianny, F. and Zernica-Goetz, M., (2000), NATURE CELL BIOLOGY Vol 2., 70-75. The mechanism behind RNA interference is still not entirely understood, but it appears that a double-stranded RNA (dsRNA) is broken into short pieces, typically 21-23 nucleotides in length, termed short interfering RNAs (siRNA). The siRNA triggers the degradation of mRNA that matches its sequence, thereby repressing expression of the corresponding gene. Discussed in Bass, B., NATURE 411:428-429 (2001) and Sharp, P. A., GENES DEV. 15:485-490 (2001). Indeed, the introduction of siRNAs into a cell can trigger RNAi in mammalian cells (Elbashir, S. M., et al. NATURE 411:494-498 (2001)), and the invention therefore contemplates the use of siRNAs to target degradation of mRNA containing a regulatable gene expression inhibitor sequence. Accordingly, a regulatable gene expression inhibitor sequence is any sequence to which an siRNA, dsRNA, ribozyme, or antisense RNA may be targeted. Similarly, short hairpin RNAs may also be used as a regulatory molecule, according to the invention. Short hairpin RNA (shRNA) is a form of hairpin RNA capable of sequence-specifically reducing expression of a target gene. A recent study established that such short hairpin activated gene silencing (which the researchers termed “SHAGging”) in a variety of normal and cancer cell lines, and in mouse cells as well as in human cells (Paddison, P. et al., GENES DEV. 16(8):948-58 (2002)). Methods of RNA interference using double-stranded DNA molecules are described in further detail infra.

Single-stranded DNA fragments may also be used as regulatory molecules, according to the invention. In specific embodiments, triplex molecules may be used to inhibit gene expression via a gene expression inhibitor element. Triplex molecules refer to single DNA strands that bind duplex DNA, thereby forming a collinear triplex molecule and preventing transcription (see, e.g., U.S. Pat. No. 5,176,996 to Hogan et al., which describes methods for making synthetic oligonucleotides that bind to target sites on duplex DNA).

The invention also contemplates using a regulatable splice acceptor site as a regulatable gene expression inhibitor sequence, particularly in embodiments of the invention wherein the vector inserts into an intron. Enhancer and suppressors of splicing may be used as regulatory molecules to regulate the activity of an inserted splice acceptor site, thereby regulating expression of the corresponding gene.

In general, it should be understood that any sequence or element that may be targeted to regulate gene expression is useful and may be used as a regulatable gene expression inhibitor element within the context of the present invention.

4. Markers

Markers include reporters, positive selection markers, and negative selection markers. A reporter is any molecule, including polypeptides as well as polynucleotides, expression of which in a cell produces a detectable signal, such as luminescence, for example. A selection molecule is any molecule, including polypeptides as well as polynucleotides, expression of which allows cells containing the gene to be identified, such as antibiotic resistance genes and fluorescent molecules, for example. A negative selection marker is any molecule, including polypeptides and polynucleotides, expression of which inhibits cells containing the gene to be identified, such as the HSV-tk gene, for example. Exemplary markers are disclosed in U.S. Pat. No. 5,464,764 and No. 5,625,048, which are incorporated by reference in their entirety. Procedure for selecting and detecting markers are widely available and published in the art, including, for example, in Joyner, A. L., GENE TARGETING: A PRACTICAL APPROACH, 2nd ed., (2000), Oxford University Press, New York, N.Y.

Examples of reporter genes widely used in detecting the presence of a vector include the E. coli β-galactosidase gene (lacZ), which is detected using an enzymatic assay with X-gal, the human placental alkaline phosphatase gene (HPAP), which is detected by an enzymatic assay using a substrate such as BM Purple AP Substrate (Boehringer Mannheim), and green fluorescent protein (GFP), and variants thereof (e.g. EGFP (Clontech Inc.), EYFP, and ECFP), which can be detected microscopically or by fluorescence activated cell sorting (FACS). In addition, glucose phosphate isomerase (GPI) may be used as a marker to detect chimeras by GPI cellulose-acetate electrophoresis.

A variety of different selection/selectable marker genes are available in the art to identify vector integration into genomic DNA. Selectable markers that may be used according to the invention, include, for example, dominant and negative section markers, as well as positive and negative selection markers. Examples of preferred selectable markers include neomycin phosphotransferase (neo), histidinol dehydrogenase (hisD), hygromycin resistance (hygro), thymidine kinase, blasticidin S deaminase (bsr) and puromycin-N-acetyltransferase (puro). Exemplary markers also include chloramphenicol-acetyl transferase (CAT), dihydrofolate reductase (DHFR), and β-galactosyltransferase. For a list of other mammalian selection markers, see Sambrook, J., et al., MOLECULAR CLONING: A LABORATORY MANUAL, 2nd ed. (2001), Cold Spring Harbor Laboratory Press, Cold Spring Harbor, N.Y. Methods of detecting a suitable selectable marker are available in the art and depend, in part, on the origin of the targeted cell.

Other appropriate selectable marker genes include fusion proteins comprising reporter and selector proteins, particularly in-frame fusions between lacZ and selector genes, such as the βgeo marker. For example, the marker comprising an in-frame fusion of lacZ and neo (βgeo) permits direct selection of vector integrations into expressed genes, since G418 resistant colonies will only be obtained when the integration leads to the generation of a functional lacZ/neo or neo/lacZ fusion protein. Furthermore, selectable markers include cell surface proteins, including cell adhesion molecules, such as integrins. Preferably, such cell surface protein markers are not expressed or expressed at low levels in target cells.

In certain embodiments, a vector of the invention may contain a negative selection marker, such as those disclosed in U.S. Pat. No. 5,464,764, hereby incorporated by reference in its entirety. Negative selection methods typically involve removing cells that express the negative selection marker by, for example, killing them, sorting them based on fluorescence, or removing them by panning. Examples of negative selection markers that may be used according to the invention include xanthine/guanine phosphoribosyl transferase (gpt), herpes simplex thymidine kinase (HSVtk), and diptheria toxin A fragment (DTA). For example, hypoxanthine-guanine phosphoribosyltransferase (Hprt) may be used in combination with Hprt-defective cells, while the bacterial guanine/xanthine phosphoribosyltransferase (gpt) permits growth on MAX medium (Song, K. Y., et al. (1987) PROC. NAT'L ACAD. SCI. U.S.A. 84, 6820-6824). When included within a vector of the invention, negative selection markers are generally included in addition to a reporter or positive selection marker.

Preferably, endogenous promoters within genomic sequences drive marker gene expression. Alternatively, an exogenous promoter capable of driving marker gene expression may be included within the vector. In some instances, it may be preferable to include an exogenous promoter capable of driving marker gene selection to ensure that the marker gene is expressed at levels adequate for detection or selection. For example, when it is known that a homologous recombination target gene possesses a weak or inactive promoter, an exogenous promoter may be provided to drive marker gene expression, thereby facilitating identification or selection. However, if the invention is being employed to detect transcriptionally active genes, for example, it may be preferable to not include an exogenous marker gene, so that marker gene expression occurs only when the marker gene integrates into a transcriptionally active gene. Promoters that may be used to drive expression of a reporter or selector gene are widely known and available in the art and include, for example, both mammalian and viral promoters, such as thymidine kinase promoters and cytomegalovirus promoters.

5. Recombinase Recognition Sites and Recombinases

In certain embodiments, vectors of the invention comprise two recombinase recognition sites. Preferably, these recombinase recognition sites flank the marker sequence, one located 5′ of the marker sequence and the second located 3′ of the marker sequence. In preferred embodiments, the recombinase recognition sites are positioned to direct recombinase-mediated deletion of the marker sequence following identification or selection of desired integration events.

Suitable recombinase sites include FRT sites and loxP sites, which are recognized by the flp and cre recombinases, respectively (See U.S. Pat. No. 6,080,576, No. 5,434,066, and No. 4,959,317). The Cre-loxP and Flp-FRT recombinase systems are comprised of two basic elements: the recombinase enzyme and a small sequence of DNA that is specifically recognized by the particular recombinase. Both systems are capable of mediating the deletion, insertion, inversion, or translocation of associated DNA, depending on the orientation and location of the target sites. Recombinase systems are disclosed in U.S. Pat. No. 6,080,576, No. 5,434,066, and No. 4,959,317, and methods of using recombinase systems for gene disruption or replacement are provided in Joyner, A. L., Stricklett, P. K. and Torres, R. M. and Kuhn, R. In LABORATORY PROTOCOLS FOR CONDITIONAL GENE TARGETING (1997), Oxford University Press, New York.

Representative minimal target sites for Cre and Flp are each 34 base pairs in length and are known in the art. The orientation of two target sites relative to each other on a segment of DNA directs the type of modification catalyzed by the recombinase: directly orientated sites lead to excision of intervening DNA, while inverted sites cause inversion of intervening DNA. In certain embodiments, mutated recombinase sites may be used to make recombination events irreversible. For example, each recombinase target site may contain a different mutation that does not significantly inhibit recombination efficiency when alone, but nearly inactivates a recombinase site when both mutations are present. After recombination, the regenerated recombinase site will contain both mutations, and subsequent recombination will be significantly inhibited.

Recombinases useful in the present invention include, but are not limited to, Cre and Flp, and functional variants thereof, including, for example, FlpL, which contains an F70L mutation, and Flpe, which contains P2S, L33S, Y108N, and S294P mutations. Cre or Flpe is preferably used in ES cells, since they have been shown to excise a chromosomal substrate in ES cells more efficiently than FlpL or Flp (Jung, S., Rajewsky, K, and Radbruch, A., (1993), SCIENCE, 259, 984).

B. Gene Knockout Methods, Cells, and Animals

1. Disruption of a Gene

In certain embodiments, a vector of the invention is inserted into the genome of a eukaryotic target cell. In different embodiments, target cells of the invention are primary cells, cell lines, immortalized cells, or transformed cells. A target cell may be a somatic cell or a germ cell. The target cell may be a non-dividing cell, such as a neuron, or it may be capable of proliferating in vitro in suitable cell culture conditions. Target cells may be normal cells, or they may be diseased cells, including those containing a known genetic mutation. Eukaryotic target cells of the invention include mammalian cells, such as, for example, a human cell, a murine cell, a rodent cell, and a primate cell. In one embodiment, a target cell of the invention is a stem cell, which includes, for example, an embryonic stem cell, such as a murine embryonic stem cell.

The gene disrupted by the methods of the invention may be a known gene target or it may be an unknown or random gene disrupted, for example, by the insertion of a gene trap construct of the invention. In certain embodiment, it is preferable to randomly disrupt genes, for example, to identify new genes associated with specific phenotypes, disease, or cellular activities. Indeed, in certain embodiments, the invention contemplates the disruption of a plurality of genes using gene trap vectors, and in certain embodiments, the invention contemplates disrupting all genes within a genome. Thus, the invention includes methods of generating libraries containing a plurality of cells, each containing one or more different gene disruptions, and the libraries generated according to these methods. In one embodiment, these libraries are produced by introducing a gene trap vector of the invention into a plurality of cells, wherein the vector inserts into different sites into the genome of different cells, thereby disrupting a plurality of genes within the library.

Vectors of the invention are introduced into a cell by any means available in the art, including, for example, electroporation, microinjection, transfection, infection, lipofection, gene gun, and retrotransposition. Generally, a suitable method of introducing a vector into a cell is readily determined by one of skill in the art based upon the type of vector and the type of cell, and teachings widely available in the art.

Cells containing vector sequences integrated within the cellular genome are selected by any means available in the art. In one preferred embodiment, cells containing vector sequence within an untranslated region of a gene are identified based upon expression of the marker. Preferably, the marker is a positive selection marker that allows the selection of cells containing insertions within an untranslated region of a gene. Cells containing integrated vector sequences may also be selected based upon expression of a negative selection marker when such a marker is present in the vector. Selection is accomplished according to methods available in the art for the particular selection marker. For example, selection may be accomplished by growing cells transfected with a vector containing a positive selection marker in selective media that permits cell growth only when the positive selection marker is expressed. Integration events may also be identified and confirmed by routine molecular biology techniques, including southern blotting and sequencing portions of the integrated vector sequences and surrounding genomic sequence. Similarly, the identity of a gene disrupted by vector sequence insertion may be determined by sequencing genomic DNA surrounding the inserted vector sequence. Methods of obtaining genomic DNA and DNA sequencing are routine and known in the art.

Target cells may contain integrated vector sequence in one or more alleles of a disrupted gene. In certain embodiments, following selection, the target cell will contain disruptions in both or all alleles of a disrupted gene. Cells containing disruption of both or all alleles of a gene may be produced by sequentially disrupting each allele, or by utilizing a selection procedure that preferably selects for cells wherein both alleles are disrupted. For example, wherein a selectable marker confers resistance to a drug upon a target cell, cells containing disruption of both or all alleles may be selected by using an increased concentration of the drug. In one embodiment of the invention, a first allele of a gene is disrupted by insertion of a gene trap or targeting vector, and a second allele of the same gene is disrupted by subsequent insertion of a targeting vector. When the cassette comprising the selection marker of the first insertion vector is removed via excision using site-specific recombinases prior to disruption of a second allele, it is possible to re-use in the second insertion vector the same selection marker that was used in the first selection marker. Alternatively, a different selection marker may be used to disrupt different alleles, e.g. neo, hygro, puro, etc.

In certain embodiments, the invention provides methods of disrupting the expression of a gene by inserting vector sequences of the invention into the gene. A full length vector or a portion of a vector may be inserted according to the method. In one embodiment, the expression of a specific gene is disrupted by the insertion of targeting vector sequence into the gene, wherein the genomic sequences within the targeting vector correspond to regions of the gene. Thus, methods of the invention may be used to specifically disrupt the expression of any identified gene, provided the sequence of at least a region of the gene is known. Disruption of gene expression preferably results in an absence of expression of the gene. Expression of a disrupted gene may be reduced by any of a number of means, depending upon the vector insertion site and sequences. For example, a vector that inserts into an untranslated 5′ exon region of a gene may terminate transcription before transcribing the downstream disrupted gene, or it may transcribe a region of the disrupted gene that is not in the correct reading frame, for example.

In another embodiment, the expression of an unknown or random gene is disrupted by inserting at least a portion of a gene trap vector into the gene. In a related embodiment, a library of randomly mutated cells is generated by introducing a gene trap vector of the invention into a multitude of cells to produce a multitude of randomly mutated cells. As used herein, disruption of a gene includes the disruption of one or more alleles of the gene.

In related embodiments, one allele of a random or unknown gene is disrupted by the insertion of at least a portion of a gene trap vector in to the allele, and a second allele of the same gene is subsequently disrupted by insertion of at least a portion of a targeting vector. Similarly, a gene of interest may be originally identified by a method comprising the insertion of a gene trap vector into one allele of the gene, and subsequently, one or more alleles of the gene may be disrupted by the insertion of at least a portion of a targeting vector into the same gene in the same or a different cell.

The invention also provides methods of disrupting expression of a gene is an animal. Methods for obtaining transgenic and knockout animals are well known in the art. Methods of generating a mouse containing an introduced gene disruption are described, for example, in Hogan, B. et al., (1994), MANIPULATING THE MOUSE EMBRYO: A LABORATORY MANUAL, 2nd ed., Cold Spring Harbor Laboratory Press, Cold Spring Harbor, N.Y. and Joyner. In one embodiment, gene targeting, which is a method of using homologous recombination to modify a cell's or animal's genome, can be used to introduce changes into cultured embryonic stem cells. By targeting a target gene of interest in ES cells, these changes can be introduced into the germlines of animals to generate chimeras and knock-out animals.

Generally, the ES cells used to produce the knockout animals will be of the same species as the knockout animal to be generated. Thus, for example, mouse embryonic stem cells are used for generation of knockout mice. Embryonic stem cells are generated and maintained using methods well known in the art such as those described, for example, in Doetschman, T., et al., J. Embryol. Exp. Morphol. 87:27-45 (1985), and improvements thereof. Any line of ES cells may be used according to the invention. However, the line chosen is typically selected for the ability of the cells to integrate into and become part of the germ line of a developing mouse embryo so as to create germ line transmission of the knockout construct. One example of a mouse strain commonly used for production of ES cells is the 129J strain. Other examples include the murine cell line D3 (American Type Culture Collection, catalog no. CKL 1934) and the WW6 cell line (loffe, et al., PNAS 92:7357-7361). The cells are cultured and prepared for knockout construct insertion using methods well known to one of ordinary skill in the art, such as those set forth by Robertson in: TERATOCARCINOMAS AND EMBRYONIC STEM CELLS: A PRACTICAL APPROACH, E. J. Robertson, ed. IRL Press, Washington, D.C. (1987); by Bradley et al., CURRENT Topics IN DEVEL. BIOL. 20:357-371 (1986); and by Hogan et al., MANIPULATING THE MOUSE EMBRYO: A LABORATORY MANUAL, Cold Spring Harbor Laboratory Press, Cold Spring Harbor, N.Y. (1986).

Briefly, in certain embodiments, a gene is disrupted in embryonic stem cells, and cells containing the knockout construct in the proper location are identified by selection techniques, as described above. After suitable ES cells have been identified, the cells may be inserted into an embryo. Insertion may be accomplished in a variety of ways known to the skilled artisan, however, a preferred method is microinjection. The cell may be injected into preimplantation embryos (typically blastocysts). The suitable stage of development for the embryo used for insertion of ES cells is very species dependent, however, for mice, one appropriate age is 3.5 days.

Following introduction of the cells into an embryo, the embryo may be implanted into the uterus of a pseudopregnant foster mother for gestation. Alternatively, the ES cells may be aggregated with morula stage embryos to produce mice. Germline chimeras are selected according to methods well known in the art, and animals homozygous for the disruption are generated by mating. According to preferred methods of the invention, an ES cell is treated with a recombinase prior to injection into a preimplantation embryo, so as to allow normal expression of the disrupted gene and prevent embryonic lethality that might result from a lack of expression of the disrupted gene. Animals of the invention include all species. Preferred animals of the invention include mice, humans, primates, rats, chickens, pigs, sheep, and cows. Other preferred methods of the invention include those methods described in PCT application WO/0042174 and PCT application WO/0051424, including double nuclear transfer.

In another embodiment, the invention provides cells and animals containing, within an endogenous gene, vector sequences that include a regulatable gene expression inhibition element and a marker cassette comprising, in operable combination, two recombinase target sites flanking a marker. In preferred embodiments, the cell or animal expresses reduced levels of the polypeptide expressed by the corresponding wild type gene. In more preferred embodiments, the cell or animal does not express any polypeptides encoded by the wild type gene. In one embodiment, a single allele of a gene is disrupted, while in another embodiment, both or all alleles of a gene are disrupted.

Typically, transgenic animals of the invention include within some or a plurality of their cells a regulatable gene expression inhibitor element, through which the expression of an endogenous gene may be repressed or lessened.

2. Generating a Conditional Knockout

The invention further provides a method of restoring expression of a gene disrupted by insertion of vector sequences of the invention, as described above. The method essentially involves delivering an appropriate recombinase to the effected cell or animal, so that the recombinase specifically recognizes the recombinase target sites introduced into the genome and excises intervening polynucleotide sequence, including the marker. If the recombinase recognition sites are loxP sites, then the appropriate recombinase is cre, or a variant thereof, whereas, if the recombinase recognition sites are FRT sites, then the appropriate recombinase is flp, or a variant thereof. Excision of the marker sequence leaves essentially only the regulatable gene expression inhibition element and a recreated wild type or mutant recombinase target site within an untranslated region of gene and restores expression of the gene. Preferably, restored levels of gene expression are approximately identical or similar to wild type expression levels. For example, restored expression levels may be at least 20%, 50%, 70%, 80%, or, preferably, greater than 90% (including any integer value between 20% and 100%), as compared to levels of wild type expression. Methods of determining mRNA or polypeptide expression levels are known in the art and include, for example, quantitative PCR, RT-PCR, northern blotting, primer extension, S1 nuclease protection, western blotting, immunoprecipitation, and immunofluorescence.

A recombinase may be introduced into a cell or animal by any means available in the art, including, for example, transfection, electroporation, scrape-loading or infection with a vector expressing the recombinase, or microinjection. Suitable cre expression vectors include pIC-Cre and pMC-Cre, described in Gu, H. et al., (1993), Cell, 73, 1155 and Torres, R. M. and Kuhn, R., (1997), In LABORATORY PROTOCOLS FOR CONDITIONAL GENE TARGETING, (R. M. Torres and R. Kuhn, eds.), Oxford University Press, Oxford, respectively. Flp expression vectors, including pOG-Flpe and phACTB-Flpe, are described in Buchholz, F. et al., (1998), Nature Biotechnol., 16, 657. Cre recombinase is also commercially available from Novagen (Madison, Wis.) and Stratagene (La Jolla, Calif.), for example, and flp recombinase expression vectors are commercially available from Stratagene. In certain embodiments, the recombinase is delivered transiently, although other embodiments contemplate constitutive delivery of the recombinase, for example, by integration of a polynucleotide that expresses the transgene into a cell or animal genome. Protocols for delivering recombinases are available in the art, including, for example, in Joyner at p. 51.

In certain methods of generating a conditional knockout animal, an embryonic stem cell containing a disrupted gene is treated with a recombinase prior to the production of chimeras or implantation into an animal. This procedure is particularly advantageous when a disrupted gene is required for embryonic development, as it allows approximately normal gene expression following treatment with the appropriate recombinase. In another embodiment, the recombinase is delivered after the generation of an animal containing at least one disrupted gene allele, by mating the animal containing a disrupted gene with an animal expressing the recombinase. The animal expressing the recombinase may express it ubiquitously, or its expression may be tissue-restricted or temporal-restricted, for example.

Thus, the invention includes methods of generating a conditional knockout cell or animal. In certain embodiments, these methods include introducing a vector of the invention into a gene within a cell or animal, followed by introducing a recombinase into the cell or animal, whereby the recombinase mediates the excision of marker sequence and restores approximately normal gene expression of the disrupted gene, under permissive conditions. Permissive conditions are conditions whereby a regulatory molecule is not causing significantly reduced expression via a regulatable gene expression inhibitor element within the disrupted gene. A recombinase may be introduced to an animal of the invention by mating, for example, by mating an animal containing a marker cassette of the invention with an animal expressing a recombinase capable of binding the recombinase sites within the marker cassette. A description of such methods is provided, for example, in WO 99/53017 A3, which is hereby incorporated by reference in its entirety.

The invention also provides knockout cells and animals containing within an endogenous gene, a regulatable gene expression inhibition element that permits conditional or selective inhibition of the gene by a regulatory molecule capable of inhibiting gene expression via the regulatable gene expression inhibitor element. Generally, these conditional knockout cells and animals also contain a functional or mutant recombinase target site within the disrupted gene, which was created upon recombinase excision of the marker and other sequence. In preferred embodiments, expression of the disrupted gene within a cell or animal of the invention is approximately normal in the absence of an active regulatory molecule capable of altering expression of the disrupted gene via the gene expression inhibitor element. In preferred embodiments, the regulatable gene expression inhibitor element is not present within the normal gene corresponding to the disrupted gene. In other preferred embodiments, the regulatable gene expression inhibitor element is not present elsewhere within the genome of the cell or animal containing the disrupted gene. Similarly, in preferred embodiments, regulatory molecules capable of regulating expression through a regulatable gene expression inhibitor element are not normally present within a cell or animal. In most preferred embodiments, regulatory molecules do not significantly effect the expression of a non-disrupted gene in the target cell or animal.

3. Regulating Gene Expression

The invention provides a variety of methods for conditionally inhibiting expression of a disrupted gene containing a regulatable gene expression inhibitor element. Expression of the disrupted gene can be regulated in any of a number of ways, according to the invention. Typically, gene expression is inhibited by either delivering or activating an inhibitory regulatory molecule that acts through the regulatable gene expression inhibitor element present in the disrupted gene. In certain embodiments, the inhibitory regulatory molecule inhibits expression directly, for example, by binding to the gene expression inhibitory element. In other embodiment, the inhibitory regulatory molecule inhibits expression indirectly. For example, the invention contemplates inhibitory regulatory molecules that function as binding partners, activator, or regulators of other molecules that act directly on the gene expression inhibition element to prevent gene expression. In yet other embodiments, a regulatory molecule acts by preventing another regulatory molecule from inhibiting gene expression. In these embodiments, removal of the regulatory molecule from the cell or animal results in a decrease in expression of the disrupted gene. Therefore, regulatory molecules of the invention include both molecules that regulate the expression of the disrupted gene and molecules that regulate the expression or activity of another regulatory molecule that regulates the expression of the gene. Thus, the invention contemplates the use of multiple layers of regulation, including cascades of regulatory molecule activation, to achieve conditional regulation of the expression of a disrupted gene. A regulatory molecule according to the invention is any molecule capable of altering expression of a disrupted gene via the introduced regulatable gene expression inhibitor element.

Regulatory molecules may be delivered to a cell by means available in the art, including, for example, electroporation, transfection, infection, transport by cell receptors, diffusion across a cell membrane, or microinjection. In addition, the regulatory molecule may be delivered by causing its transport into the nucleus from the cytoplasm, another organelle, or the cell or nuclear membrane, for example. In preferred embodiments, regulatory molecules are polynucleotides or polypeptides. Thus, regulatory molecules may be provided in these forms or they may be expressed from vectors introduced into a target cell or animal. The invention also contemplates providing regulatory molecules by mating an animal of the invention with a transgenic animal capable of expressing the regulatory molecule.

Furthermore, according to the invention, the expression or activity of the regulatory molecule, itself, may be regulated by another molecule. In a preferred embodiment, an expression vector capable of induced expression of the regulatory molecule is provided to a cell or animal containing a disrupted gene capable of being regulated by the regulatory molecule. The regulatory molecule expression vector may be transiently present in the cell, or it may be stably present in the cell, for example, as an episome or stably integrated into the cell's genome. Similarly, in animals containing a transgene capable of expressing the regulatory molecule, the transgene may be regulated in an inducible or tissue- or stage-specific manner, via elements located within the transgene, including transcription enhancer elements, for example.

In one embodiment of the invention, expression of the regulatory protein is regulated by a conditional promoter, and expression of the disrupted gene is regulated by inducing or inhibiting the expression of the regulatory molecule. Examples of such a system include prokaryotic repressors that can transcriptionally repress a disrupted gene into which an appropriate repressor-binding sequence has been inserted. In certain embodiments, repressors for use in the present invention are sensitive to inactivation by physiologically benign inducing agents. Thus, or example, where the lac repressor protein is used according to the invention to control the expression of a eukaryotic promoter engineered to contain a lacO operator sequence (i.e. regulatable gene expression inhibitor sequence), treatment of the host cell with IPTG will cause the dissociation of the lac repressor from the engineered promoter and allow transcription to occur. Similarly, where the tet repressor is used to control the expression of a eukaryotic promoter which has been engineered to contain a tetO operator sequence, treatment of the host cell with IPTG will cause the dissociation of the tet repressor from the engineered promoter and allow transcription of the disrupted gene.

In certain embodiments of the invention, the use of prokaryotic repressor or activator proteins is advantageous due to their specificity for a corresponding prokaryotic sequence not normally found in a eukaryotic cell. One example of this type of inducible system is the tetracycline-regulated inducible promoter system, of which various useful version have been described. See, e.g. Shockett and Schatz, PROC. NATL. ACAD. SCI. USA 93:5173-76 (1996) for a review. In one embodiment of the invention, for example, expression of the inhibitory regulatory molecule can be placed under control of the REV-TET system. Components of this system and methods of using the system to control the expression of a gene are well-documented in the literature, and vectors expressing the tetracycline-controlled transactivator (tTA) or the reverse tTA (rtTA) are commercially available (e.g. pTet-Off, pTet-On and ptTA-2/3/4 vectors, Clontech, Palo Alto, Calif.). Such systems are described, for example, in U.S. Pat. No. 5,650,298, No. 6,271,348, No. 5,922,927, and related patents, which are incorporated by reference in their entirety.

Briefly, in certain embodiments, these vectors express fusion proteins of the VP16 transactivator (tTA or rtTA) that activate transcription in the absence or presence of doxycycline, respectively. Thus, in certain embodiments, the presence of doxycycline or tetracycline prevents expression of an inhibitory regulatory molecule. In other embodiments, the presence of doxycycline or tetracycline permits expression of an inhibitory regulatory molecule. For example, expression of an antisense RNA or ribozyme may be placed under control of a VP16 responsive promoter, and their expression regulated by the addition of doxycycline to media. Once activated, the transcribed molecules are free to associate with the target protein mRNA, leading to degradation of the mRNA. Specific REV-TET systems are described in Gossen, M. and Bujard, H. (1992) PROC NATL ACAD SCI USA 89, 5547-51 and Baron, U., Schnappinger, D., Helbl, V., Gossen, M., Hillen, W. and Bujard, H. (1999) PROC NATL ACAD SCI USA 96, 1013-1018, and references cited within.

It should be understood that the present invention allows for considerable flexibility and a wide range of suitable inducible promoters and corresponding inducing agents, when used. In some embodiments of the invention, the choice of an inducible promoter may be governed by the suitability of the required inducing agent. Factors such as cytotoxicity or indirect effects on nontarget genes may be important to consider. In other instances, the choice may be governed by the properties of the inducible system as a whole. Examples of factors that might be important to consider include the ease with which the system can be introduced into the appropriate cell and the speed and strength with which induction of the system occurs following exposure to an inducing agent. Again, it is reiterated that the particular system chosen to induce or activate an effector of repression (i.e., regulatory molecule) through a regulatable gene expression inhibitor sequence may operate in the presence of absence of an inducing agent, depending on the particular system chosen. Thus, in certain embodiments, cells will be maintained in an agent or compound to avoid repression of the disrupted gene, while in other embodiments, an agent or compound will be added to induce repression of a disrupted gene.

C. Applications of Conditional Knockout Methods, Cells, and Animals

The invention contemplates a variety of uses for cells and animals containing conditional gene knockouts. Indeed, uses of the invention are virtually unlimited, and essentially any previous known uses for gene trap vectors, homologous recombination vectors, and conditional knockouts may be addressed using the presently described vectors, cells, animals, systems, and methods. The vectors, cells, animals, systems, and methods of the invention may be used to study both basic biological processes and disease. In addition, the compositions and methods of the invention are suited for identifying the molecular basis for both disadvantageous and advantageous biological traits, such as prolonged life-span, low cholesterol, lack of obesity, and lack of susceptibility to a disease, for example.

Conditional knockout cells and animals of the invention may be used to analyze the function of a known gene, identify a gene possessing a particular function or involved in a particular cell process, identify genes involved in disease, and to generate cell and animal models of diseases, for example. In addition, the conditional knockouts may be used to identify the role of a gene at different stages of development or in different tissues, for example. The invention also provides methods of using the knockout cells and animals to analyze the function of compounds, including, for example, small molecules, and methods of screening compounds to identify new drugs or new pharmaceutical indications for known drugs. In related methods, the invention provides a means to identify a compound that inhibits or enhances the activity of a gene product. The invention also provides high throughput screening assays to identify and select for compounds that effect the activity of a gene product. In addition, the invention may be used to model the effects of drug administration, including acute administration, in a cell or animal.

In one embodiment, the invention provides a method of determining the function of a gene. In one embodiment, two knockout cells, prepared according to methods of the invention, that contain the same gene disruption are provided. Expression of the disrupted gene is reduced in one cell by introducing or activating a regulatory molecule that regulates expression of the gene via the introduced gene expression inhibitor element. Then, biological traits of the two cells are examined, and the function of the disrupted gene is determined by comparing the biological traits of the cell expressing approximately normal levels of the gene with the traits of the cell wherein the regulatory molecule was introduced or activated to reduce expression of the gene. Example of traits that may be examined include, but are not limited to, anchorage independent growth, production of angiogenic factor or other genes or polypeptides, growth factor independence, growth in low nutrients, autocrine growth, alteration of activation of signal transduction pathways (e.g. Ras, p53, growth factor receptor signaling and lipid metabolism), tumorigenesis, metastasis, and cell cycle profiles.

In a related embodiment, the function of a gene may be determined using a single conditional knockout cell of the invention. According to this method, biological traits of a cell are examined and compared before and after treatment with a regulatory molecule that reduces expression of the gene. Treatment with the regulatory molecule may be transient or constitutive, and biological traits may be examined at numerous and various time points following introduction or activation of the regulatory molecule. When treatment is transient, treatment may be very brief in duration (i.e. minutes to hours), or treatment may persist for longer time periods (i.e. 1, 2, 3, 4, or more days). In certain embodiments, traits are examined following discontinuation of treatment, preferably during or after gene expression is restored, and compared to the same traits prior to treatment or following treatment, wherein gene expression levels are reduced.

In another embodiment, the function of a gene is determined by examining the traits in knockout animals of the invention. Similar to the methods described above for cells, methods of determining gene function in one or more animals provided by the invention generally involve examining biological traits of the animal in the presence or absence of a regulatory molecule that inhibits expression of the gene via the introduced regulatable gene expression inhibitor element. Gene function may be determined in a single animal by examining biological traits before introduction or activation of a regulatory molecule, and comparing them to traits examined after treatment with the regulatory molecule that inhibits gene expression. In addition, traits may also be examined following depletion or termination of treatment with the regulatory molecule. Thus, the invention also provides methods of determining the effects of transient disruption of gene expression. Methods of the invention may be used to examine the effects of transient, long-term, or permanent gene inactivation within an entire animal, in particular tissues, or at particular times in development.

In another embodiment of the invention, gene function is determined using more than one animal by comparing biological traits between different animals containing the same gene disruption, wherein one animal was treated with the regulatory molecule to disrupt gene expression, and another animal was not treated.

The invention also provides methods of identifying a gene with a specific function. According to one embodiment, a method of identifying a gene with a specific function involves providing a multitude of conditional knockout cells of the invention, wherein different cells contain the same regulatory gene expression inhibitor element within different genes. These cells are then treated with a regulatory molecule that disrupts expression of at least one gene. The cells are then examined to identify a cell wherein the specific function was altered upon treatment. Once the cell is identified, the disrupted gene can be identified according to methods available in the art, such as sequencing of the DNA surrounding the inserted vector sequences.

In a related embodiment, the invention provides a method of testing whether a particular gene is associated with a particular function. The method involves providing a conditional knockout cell or animal wherein the gene being examined is disrupted. The cell is treated with a regulatory compound that alters expression of the gene. In preferred embodiments, expression is substantially reduced or reduced to undetectable levels. The cell is then examined to determine whether the disrupted gene is associated with the hypothesized function by comparing biological traits before and after introduction of the regulatory molecule. Biological traits of the cell that are examined are determined based upon the particular function with which the gene is hypothesized to be associated. Similarly, the duration of time during which the gene is disrupted and the time points following disruption at which traits are examined are also determined by one of skill in the art depending upon the particular function being tested.

In another embodiment, the invention provides a method of generating an animal model of disease. In one embodiment, an animal model of a disease is generated by creating a conditional knockout animal according to methods of the invention. In certain embodiments, the knockout disrupts a gene that has been implicated in disease. In preferred embodiments, decreased expression of the gene is associated with a disease. Accordingly, in certain embodiments, the knockout animal is a model for a disease known to be associated with decreased expression of a gene. In other embodiments, a knockout animal is a model for a disease known to be associated with decreased activity of a gene. In some instances, such decreased activity may be due to a mutation of the gene in patients with the gene, for example. Useful animal models of a disease will possess traits associated with the disease phenotype. Suitable animal models of a disease are determined by identifying conditional knockout animals that possess traits or characteristics associated with the disease. Where the identity of a gene associated with a disease is known, an animal model of the disease may be generated by producing a targeted disruption of the gene according to methods of the invention. Where the molecular basis of a gene is not known, an animal model of the disease is produced by generating targeted disruptions of candidate genes predicted to be associated with a disease. Alternatively, an animal model of such a disease is produced by generating random gene disruptions according to methods of the invention, generating a multitude of animals with random gene disruptions, and examining biological traits of these animals following treatment with a regulatory molecule, to identify an animal with biological traits associated with the disease.

The invention additionally provides methods of screening and identifying compounds. Candidate compounds include, but are not limited to, small molecules, (such as organic molecules, for example), peptides, polypeptides, and nucleic acids. In certain embodiments, the invention provides methods for screening or identifying a compound capable of altering the expression or function of a gene. In one embodiment, these methods involve comparing the effects of treating a cell or animal of the invention with a regulatory molecule that reduces expression of the disrupted gene to the effects of treating a cell with a candidate compound. Where the effects are identical or similar, it is likely that the candidate molecule is effecting the expression or activity of the same gene product or a biologically related gene product.

In certain embodiments, the invention provides methods for screening and identifying a compound that inhibits the expression or activity of a gene product. Characteristics and biological traits associated with a loss of disrupted gene expression are determined after treatment of a cell or animal of the invention with a regulatory molecule that reduces expression of the disrupted gene via the introduced gene expression inhibitor element. These characteristics and traits are compared to those resulting from the treatment of a cell with a candidate compound, and a candidate compound that inhibits the expression or activity of the disrupted gene is identified because it causes the same traits or characteristics as treatment with the regulatory compound.

In other embodiments, methods of the invention include contacting a knockout cell of the invention with a regulatory molecule capable of regulating the expression of the disrupted gene via the introduced gene expression inhibitor element, introducing a candidate compound to the cell, and comparing expression of the disrupted gene before and after treatment with the compound. Where expression is restored by treatment with the compound, it is likely that the compound effects the expression or activity of the regulatory molecule.

In a related embodiment, the invention provides a method of identifying a compound that compensates for the loss of expression of a disrupted gene. A knockout cell or animal of the invention is treated with a regulatory molecule that reduces expression of the disrupted gene via the introduced regulatable gene expression inhibitor sequence, a candidate compound is introduced to the cell or animal, biological traits associated with the introduction of the regulatory compound are examined before and after treatment with the compound, and these biological traits are then compared. Compounds that compensate for the loss of disrupted gene expression are associated with biological traits observed prior to treatment with the regulatory compound. Alternatively, two or more cells containing the same gene disruption are provided. At least two cells are treated with a regulatory compound that decreases expression of the disrupted gene via the introduced regulatable gene expression inhibitor element. One cell is then treated with a candidate compound. Biological traits are examined in cells prior to treatment with the regulatory compound, after treatment with the regulatory compound but before treatment with the candidate compound, and after treatment with the candidate compound. A candidate compound that compensates for the loss of disrupted gene expression shares biological traits with the cell that was not treated with the regulatory molecule.

In specific embodiments, a single candidate compound may be tested for its ability to alter the expression of a disrupted gene, the activity of a gene product, or the effect of reduced expression of a gene, whereas in other embodiments, multiple candidate compounds may be screened to identify an effective compound.

Candidate compounds may be introduced to an isolated cell, multitudes of cells, or a cell within an animal. Methods of introducing a candidate compound include those methods described for introducing a regulatory molecule to a cell, and are widely known in the art. In preferred embodiments, candidate compounds are introduced to a cell by ectopically treating the cell with a candidate compound. In certain embodiments, the candidate compound is introduced into the cell by any available means. A candidate compound of the invention may act extracellularly or intracellularly.

As described above, the present invention has important applications for cell culture and animal studies. For cell culture, it represents a means for single and large-scale gene inactivation with the ability to conditionally inactivate the genes. Such cells are used to identify genes associated with various cellular functions, such as signaling networks, biochemical properties, biophysical properties and biology. For animal studies, the present invention allows for gene inactivation at any stage of animal development. In preferred embodiments, animals are quite normal in all regards until the time the gene is inactivated. This allows methods for identifying the functions of genes at various times in development and in mature animals, thereby providing a significant advantage over simple gene inactivations. The effect of gene inactivation in animal models for diseases and disorders (or other functions) can also be examined according to methods of the invention. In addition to efficacy evaluations, the methods allow for the evaluation of toxicity of gene inactivation at any stage of development, including in mature animals. Furthermore, by turning the gene on and off, it is possible to determine how quickly efficacy is observed in animal models and how rapidly toxic effects are observed

D. Advantages of the Homologous Recombination System

The invention provides systems for generating conditional knockout cells and animals. In one embodiment, a conditional gene knockout system of the invention comprises a vector of the invention and a regulatory molecule capable of regulating gene expression through the regulatable gene expression inhibitor element of the vector. The system may further comprise a means for regulating the regulatory molecule. According to different embodiments of the invention, a means for regulating the activity of the regulatory molecule includes, for example, any means to regulate expression or activity of the regulatory molecule.

The system described has several advantages over systems presently available in the art. First, the invention provides a means to conditionally express genes where the conditional system is common for any number of genes that are desired to be examined. That is, the regulatable gene expression inhibition element for one target gene can be the same for other target genes. There is no need to find separate regulatory molecules to disrupt different mRNAs derived from different genes. This means that optimization of the conditional system needs to only be done once per primary cell or cell line. Second, the invention eliminates the need to isolate cDNA for each gene for which conditional expression following gene inactivation is desired. Third, expression of the targeted gene is not under artificial regulation. Instead, the gene is under controls that normally regulate when, where, and how much of the gene is expressed. For example, cell cycle regulation and growth factor regulation for when, tissue specific regulation and normal cellular location for where, and normal levels (per normal promoter strength, regulation, and site within the chromosome) for how much. Fourth, because cells express the gene normally, they do not adapt to abnormal location or levels by changing expression or regulation of other proteins. Hence, the cells or animals very much represent normal animals, until the gene is inactivated. Fifth, because the gene product is not chronically inactivated, the cells do not adapt over time, again by changing expression or activation states of other proteins.

E. Conditional Knockdown of Gene Expression

Conditional regulation of gene expression can also be regulated through the use of knockdown reagents, which are believed to reduce gene expression, e.g., by targeting the degradation of specific mRNA transcripts. Accordingly, the invention includes a system and components for the conditional regulation of gene expression, which involves the use of knockdown reagents directed to a target gene. Essentially, the system includes two elements, which together provide for the conditional regulation of a target gene. The first element of the system involves the knockdown of expression of an endogenous target gene. The second element of the system involves the conditional expression of a variant of the endogenous target gene, i.e., an ectopic gene variant, which is not significantly affected by the reagent used to knockdown expression of the endogenous target gene. Thus, this aspect of the invention provides a means to conditionally regulate the expression of a variant gene, typically possessing the same functional properties of the endogenous target gene and expressed polypeptide, while reducing expression of the endogenous gene via knockdown methods and reagents. In certain embodiment, this conditional knockdown system may be used independently of or in conjunction with the conditional knockdown system of the invention.

1. Knockdown Reagents

Any knockdown reagent may be used according to the invention, including not limited to, (i) antisense sequences, (ii) catalytic RNAs (ribozymes), and (iii) double-stranded RNA (dsRNA), including, for example, short interfering RNA (siRNA) and short hairpin RNA (shRNA), etc. Such knockdown reagents generally target a specific nucleotide sequence in genomic DNA or mRNA transcripts.

a. Antisense

Antisense oligonucleotides have been demonstrated to be effective and targeted inhibitors of protein synthesis, and, consequently, can be used to specifically inhibit protein synthesis by a targeted gene. The efficacy of antisense oligonucleotides for inhibiting protein synthesis is well established. For example, the synthesis of polygalactauronase and the muscarine type 2 acetylcholine receptor are inhibited by antisense oligonucleotides directed to their respective mRNA sequences (U.S. Pat. No. 5,739,119 and U.S. Pat. No. 5,759,829). Further, examples of antisense inhibition have been demonstrated with the nuclear protein cyclin, the multiple drug resistance gene (MDG1), ICAM-1, E-selectin, STK-1, striatal GABA_{A} receptor and human EGF (Jaskulski et al., Science. 1988 Jun. 10;240(4858):1544-6; Vasanthakumar and Ahmed, Cancer Commun. 1989;1(4):225-32; Peris et al., Brain Res Mol Brain Res. 1998 Jun. 15;57(2):310-20; U.S. Pat. No. 5,801,154; U.S. Pat. No. 5,789,573; U.S. Pat. No. 5,718,709 and U.S. Pat. No. 5,610,288). Furthermore, antisense constructs have also been described that inhibit and can be used to treat a variety of abnormal cellular proliferations, e.g. cancer (U.S. Pat. No. 5,747,470; U.S. Pat. No. 5,591,317 and U.S. Pat. No. 5,783,683).

Therefore, in certain embodiments, the present invention provides oligonucleotide sequences that comprise all, or a portion of, any sequence that is capable of specifically binding to a selected target polynucleotide sequence, or a complement thereof. In one embodiment, the antisense oligonucleotides comprise DNA or derivatives thereof. In another embodiment, the oligonucleotides comprise RNA or derivatives thereof. The antisense oligonucleotides may be modified DNAs comprising a phosphorothioated modified backbone. Also, the oligonucleotide sequences may comprise peptide nucleic acids or derivatives thereof. In each case, preferred compositions comprise a sequence region that is complementary, and more preferably, completely complementary to one or more portions of a target gene or polynucleotide sequence. Selection of antisense compositions specific for a given sequence is based upon analysis of the chosen target sequence and determination of secondary structure, Tₘ, binding energy, and relative stability. Antisense compositions may be selected based upon their relative inability to form dimers, hairpins, or other secondary structures that would reduce or prohibit specific binding to the target mRNA in a host cell. Highly preferred target regions of the mRNA include those regions at or near the AUG translation initiation codon and those sequences which are substantially complementary to 5′ regions of the mRNA. These secondary structure analyses and target site selection considerations can be performed, for example, using v.4 of the OLIGO primer analysis software and/or the BLASTN 2.0.5 algorithm software (Altschul et al., Nucleic Acids Res. 1997, 25(17):3389-402).

The use of an antisense delivery method employing a short peptide vector, termed MPG (27 residues), is also contemplated. The MPG peptide contains a hydrophobic domain derived from the fusion sequence of HIV gp41 and a hydrophilic domain from the nuclear localization sequence of SV40 T-antigen (Morris et al., Nucleic Acids Res. 1997 Jul. 15;25(14):2730-6). It has been demonstrated that several molecules of the MPG peptide coat the antisense oligonucleotides and can be delivered into cultured mammalian cells in less than 1 hour with relatively high efficiency (90%). Further, the interaction with MPG strongly increases both the stability of the oligonucleotide to nuclease and the ability to cross the plasma membrane.

b. Ribozymes

According to another embodiment of the invention, ribozyme molecules are used to inhibit expression of a target gene or polynucleotide sequence. Ribozymes are RNA-protein complexes that cleave nucleic acids in a site-specific fashion. Ribozymes have specific catalytic domains that possess endonuclease activity (Kim and Cech, Proc Natl Acad Sci USA. 1987 December;84(24):8788-92; Forster and Symons, Cell. 1987 Apr. 24;49(2):211-20). For example, a large number of ribozymes accelerate phosphoester transfer reactions with a high degree of specificity, often cleaving only one of several phosphoesters in an oligonucleotide substrate (Cech et al., Cell. 1981 December;27(3 Pt 2):487-96; Michel and Westhof, J Mol Biol. 1990 Dec. 5;216(3):585-610; Reinhold-Hurek and Shub, Nature. 1992 May 14;357(6374):173-6). This specificity has been attributed to the requirement that the substrate bind via specific base-pairing interactions to the internal guide sequence (“IGS”) of the ribozyme prior to chemical reaction.

At least six basic varieties of naturally-occurring enzymatic RNAs are known presently. Each can catalyze the hydrolysis of RNA phosphodiester bonds in trans (and thus can cleave other RNA molecules) under physiological conditions. In general, enzymatic nucleic acids act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

The enzymatic nature of a ribozyme may be advantageous over many technologies, such as antisense technology (where a nucleic acid molecule simply binds to a nucleic acid target to block its translation), since the concentration of ribozyme necessary to affect inhibition of expression is lower than that of an antisense oligonucleotide. This advantage reflects the ability of the ribozyme to act enzymatically. Thus, a single ribozyme molecule is able to cleave many molecules of target RNA. In addition, the ribozyme is a highly specific inhibitor, with the specificity of inhibition depending not only on the base pairing mechanism of binding to the target RNA, but also on the mechanism of target RNA cleavage. Single mismatches, or base-substitutions, near the site of cleavage can completely eliminate catalytic activity of a ribozyme. Similar mismatches in antisense molecules do not prevent their action (Woolf et al., Proc Natl Acad Sci U S A. 1992 Aug. 15;89(16):7305-9). Thus, the specificity of action of a ribozyme is greater than that of an antisense oligonucleotide binding the same RNA site.

The enzymatic nucleic acid molecule may be formed in a hammerhead, hairpin, a hepatitis δ virus, group I intron or RNaseP RNA (in association with an RNA guide sequence) or Neurospora VS RNA motif, for example. Specific examples of hammerhead motifs are described by Rossi et al. Nucleic Acids Res. 1992 Sep. 11;20(17):4559-65. Examples of hairpin motifs are described by Hampel et al (Eur. Pat. Appl. Publ. No. EP 0360257), Hampel and Tritz, Biochemistry 1989 Jun. 13;28(12):4929-33; Hampel et al., Nucleic Acids Res. 1990 Jan. 25;18(2):299-304 and U.S. Pat. No. 5,631,359. An example of the hepatitis δ virus motif is described by Perrotta and Been, Biochemistry. 1992 Dec. 1;31(47):11843-52; an example of the RNaseP motif is described by Guerrier-Takada et al., Cell. 1983 December;35(3 Pt 2):849-57; Neurospora VS RNA ribozyme motif is described by Collins (Saville and Collins, Cell. 1990 May 18;61(4):685-96; Saville and Collins, Proc Natl Acad Sci USA. 1991 Oct. 1;88(19):8826-30; Collins and Olive, Biochemistry. 1993 Mar. 23;32(11):2795-9); and an example of the Group I intron is described in (U.S. Pat. No. 4,987,071). Important characteristics of enzymatic nucleic acid molecules used according to the invention are that they have a specific substrate binding site which is complementary to one or more of the target gene DNA or RNA regions, and that they have nucleotide sequences within or surrounding that substrate binding site which impart an RNA cleaving activity to the molecule. Thus the ribozyme constructs need not be limited to specific motifs mentioned herein.

Ribozymes may be designed as described in Int. Pat. Appl. Publ. No. WO 93/23569 and Int. Pat. Appl. Publ. No. WO 94/02595, each specifically incorporated herein by reference and synthesized to be tested in vitro and in vivo, as described. Such ribozymes can also be optimized for delivery. While specific examples are provided, those in the art will recognize that equivalent RNA targets in other species can be utilized when necessary.

Ribozyme activity can be optimized by altering the length of the ribozyme binding arms or chemically synthesizing ribozymes with modifications that prevent their degradation by serum ribonucleases (see e.g., Int. Pat. Appl. Publ. No. WO 92/07065; Int. Pat. Appl. Publ. No. WO 93/15187; Int. Pat. Appl. Publ. No. WO 91/03162; Eur. Pat. Appl. Publ. No. 92110298.4; U.S. Pat. No. 5,334,711; and Int. Pat. Appl. Publ. No. WO 94/13688, which describe various chemical modifications that can be made to the sugar moieties of enzymatic RNA molecules), modifications which enhance their efficacy in cells, and removal of stem II bases to shorten RNA synthesis times and reduce chemical requirements.

c. Double-Stranded RNA

RNA interference methods using double-stranded RNA also may be used to disrupt the expression of a gene or polynucleotide of interest. A dsRNA molecule that targets and induces degradation of an mRNA that is derived from a gene or polynucleotide of interest can be introduced into a cell. The exact mechanism of how the dsRNA targets the mRNA is not essential to the operation of the invention, other than the dsRNA shares sequence homology with the mRNA transcript. The mechanism could be a direct interaction with the target gene, an interaction with the resulting mRNA transcript, an interaction with the resulting protein product, or another mechanism. Again, while the exact mechanism is not essential to the invention, it is believed the association of the dsRNA to the target gene is defined by the homology between the dsRNA and the actual and/or predicted mRNA transcript. It is believed that this association will affect the ability of the dsRNA to disrupt the target gene. DsRNA methods and reagents are described in PCT application WO 01/68836, WO 01/29058, WO 02/44321, and WO 01/75164, which are hereby incorporated by reference in their entirety.

In one embodiment of the invention, double-stranded RNA interference (dsRNAi) may be used to specifically inhibit target nucleic acid expression. Briefly, it is hypothesized that the presence of double-stranded RNA dominantly silences gene expression in a sequence-specific manner by causing the corresponding RNA to be degraded. Although first discovered in lower organisms such as the nematode and Drosphila, for example, dsRNAi has also been demonstrated to work in fungi, plants, and mammalian cells (Wianny, F. and Zernica-Goetz, M. (2000), Nature Cell Biology Vol. 2, 70-75). However, transfection of long dsRNAs into mammalian cells can result in nonspecific gene suppression, as opposed to the gene-specific suppression observed in other organisms.

Although the mechanisms behind dsRNAi is still not entirely understood, experiments demonstrated that, in the cell, a double-stranded RNA (dsRNA) is cleaved into short pieces, typically 21-25 nucleotides in length, termed short interfering RNAs (siRNAs), by a ribonuclease such as DICER. The siRNAs subsequently assemble with protein components into an RNA-induced silencing complex (RISC), which binds to and tags the complementary portion of the target mRNA for nuclease digestion. The siRNA triggers the degradation of mRNA that matches its sequence, thereby repressing expression of the corresponding gene. Discussed in Bass, B. Nature 411:428-429 (2001) and Sharp, P. A. Genes Dev. 15:485-490 (2001).

Double-stranded RNA-mediated suppression of gene and nucleic acid expression may be accomplished according to the invention by introducing dsRNA, siRNA or shRNA into cells or organisms. dsRNAs less than 30 nucleotides in length do not appear to induce nonspecific gene suppression, as described above for long dsRNA molecules. Indeed, the direct introduction of siRNAs to a cell can trigger RNAi in mammalian cells (Elshabir, S. M., et al. Nature 411:494-498 (2001)). Furthermore, suppression in mammalian cells occurred at the RNA level and was specific for the targeted genes, with a strong correlation between RNA and protein suppression (Caplen, N. et al., Proc. Natl. Acad. Sci. USA 98:9746-9747 (2001)). In addition, it was shown that a wide variety of cell lines, including HeLa S3, COS7, 293, NIH/3T3, A549, HT-29, CHO-KI and MCF-7 cells, are susceptible to some level of siRNA silencing (Brown, D. et al. TechNotes 9(1):1-7, available at http://www.ambion.com/techlib/tn/91/912.html (Sep. 1, 2002)).

Structural characteristics of effective siRNA molecules have been identified. Elshabir, S. M. et al. (2001) Nature 411:494-498 and Elshabir, S. M. et al. (2001), EMBO 20:6877-6888. Accordingly, one of skill in the art would understand that a wide variety of different siRNA molecules may be used to target a specific gene or transcript. In certain embodiments, siRNA molecules according to the invention are 18-25 nucleotides in length, including each integer in between. In one embodiment, an siRNA is 21 nucleotides in length. In certain embodiments, siRNAs have 0-7 nucleotide 3′ overhangs or 0-4 nucleotide 5′ overhangs. In one embodiment, an siRNA molecule has a two nucleotide 3′ overhang. In one embodiment, an siRNA is 21 nucleotides in length with two nucleotide 3′ overhangs (i.e. they contain a 19 nucleotide complementary region between the sense and antisense strands). In certain embodiments, the overhangs are UU or dTdT 3′ overhangs. Generally, siRNA molecules are completely complementary to one strand of a target DNA molecule, since even single base pair mismatches have been shown to reduce silencing. In other embodiments, siRNAs may have a modified backbone composition, such as, for example, 2′-deoxy- or 2′-O-methyl modifications. However, in preferred embodiments, the entire strand of the siRNA is not made with either 2′ deoxy or 2′-O-modified bases.

In one embodiment, siRNA target sites are selected by scanning the target mRNA transcript sequence for the occurrence of AA dinucleotide sequences. Each AA dinucleotide sequence in combination with the 3′ adjacent approximately 19 nucleotides are potential siRNA target sites. In one embodiment, siRNA target sites are preferentially not located within the 5′ and 3′ untranslated regions (UTRs) or regions near the start codon (within approximately 75 bases), since proteins that bind regulatory regions may interfere with the binding of the siRNP endonuclease complex (Elshabir, S. et al. Nature 411:494-498 (2001); Elshabir, S. et al. EMBO J. 20:6877-6888 (2001)). In addition, potential target sites may be compared to an appropriate genome database, such as BLAST, available on the NCBI server at www.ncbi.nlm, and potential target sequences with significant homology to other coding sequences eliminated.

Short hairpin RNAs may also be used to inhibit or knockdown gene or nucleic acid expression according to the invention. Short Hairpin RNA (shRNA) is a form of hairpin RNA capable of sequence-specifically reducing expression of a target gene. Short hairpin RNAs may offer an advantage over siRNAs in suppressing gene expression, as they are generally more stable and less susceptible to degradation in the cellular environment. It has been established that such short hairpin RNA-mediated gene silencing (also termed SHAGging) works in a variety of normal and cancer cell lines, and in mammalian cells, including mouse and human cells. Paddison, P. et al., Genes Dev. 16(8):948-58 (2002). Furthermore, transgenic cell lines bearing chromosomal genes that code for engineered shRNAs have been generated. These cells are able to constitutively synthesize shRNAs, thereby facilitating long-lasting or constitutive gene silencing that may be passed on to progeny cells. Paddison, P. et al., Proc. Natl. Acad. Sci. USA 99(3):1443-1448 (2002).

ShRNAs contain a stem loop structure. In certain embodiments, they may contain variable stem lengths, typically from 19 to 29 nucleotides in length, or any number in between. In certain embodiments, hairpins contain 19 to 21 nucleotide stems, while in other embodiments, hairpins contain 27 to 29 nucleotide stems. In certain embodiments, loop size is between 4 to 23 nucleotides in length, although the loop size may be larger than 23 nucleotides without significantly affecting silencing activity. ShRNA molecules may contain mismatches, for example G-U mismatches between the two strands of the shRNA stem without decreasing potency. In fact, in certain embodiments, shRNAs are designed to include one or several G-U pairings in the hairpin stem to stabilize hairpins during propagation in bacteria, for example. However, complementarity between the portion of the stem that binds to the target mRNA (antisense strand) and the mRNA is typically required, and even a single base pair mismatch is this region may abolish silencing. 5′ and 3′ overhangs are not required, since they do not appear to be critical for shRNA function, although they may be present (Paddison et al. (2002) Genes & Dev. 16(8):948-58).

SiRNAs and shRNAs may be prepared by any available means, including chemical synthesis and in vitro transcription, according to standard procedures well known and available in the art. For example, in vitro transcription can be used to convert a pair of DNA oligonucleotides into an siRNA using the Silencer™ siRNA Construction Kit (Ambion). In one report, it was shown that the optimal concentration for transfection of in vitro transcribed siRNA was consistently at least 10 fold lower that that reported for chemically synthesized RNA (Elshabir, et al. (2001)). It has also been reported that chemically synthesized siRNA provided the greatest level of gene specific silencing when used at a concentration of 100-200 nM, while the same level of suppression was observed using as little as 5 nM of the in vitro transcribed siRNA (Brown, D. et al., TechNotes 9(1), available at www.ambion.com/techlib/tn/91/912.html). The optimal amount of siRNA used according to the invention will depend on a variety of factors, including, for example, the quality and purity of the RNA, the type of cell, the method of delivery, and the level of expression of the targeted nucleic acid sequence. The optimal amount of siRNA to be used for any application of the invention can be routinely determined by testing various parameters using standard techniques available in the arts. For example, the effectiveness of a particular siRNA protocol in reducing target nucleic acid expression may be determined by real-time RT-PCR using oligonucleotides specific for the targeted mRNA transcript or by western analysis using an antibody specific for the polypeptide expressed from the targeted nucleic acid sequence.

d. Expression of Knockdown Reagents

Knockdown reagents may be introduced into a cell or animal by any available means including injection, transfection, and infection, etc. Plasmid and other types of vectors may also be used to express knockdown reagents, including siRNAs and shRNAs, for example, in mammalian and other cells, as described, for example, in Brummelkamp, T. R. et al. (2002), Science 296:550-553; Paddison, P. J. et al. (2002) Genes & Dev. 16:948-958; Paul, C. P. (2002) Nature Biotechnol. 20:505-508; Sui, G. et al. (2002) Proc. Natl. Acad. Sci USA 99(6):5515-5520; Yu, J-Y, et al. (2002) Proc. Natl. Acad. Sci USA 99(9):6047-6052; Miyagishi, M. and Taira, K. (2002) Nature Biotechnol. 20:497-500; and Lee, N. S. et al. (2002) Nature Biotechnol. 20:500-505. While transfection of siRNAs into cells can transiently knock down expression of specific genes, expression of siRNA and shRNA molecules within a cell permits long term silencing. Expression of siRNA and shRNA molecules may be accomplished transiently, or stable cell lines may be established. Such stable cell lines may contain an expression cassette integrated into the cellular genome, from which siRNA or shRNA molecules are expressed.

Typically, the integrated expression cassette will comprise a promoter, but, alternatively, the siRNA or shRNA molecule may be expressed from an endogenous promoter. Suitable promoters are known in the art and include, for example, polI, polII and polIII promoters. Essentially any promoter active in a target cell may be used according to the invention. In certain embodiments, expression vectors contain either the polymerase III H1-RNA or U6 promoter.

Vectors may also contain a transcription termination signal, such as, for example, a 4-5-thymidine transcription termination signal or a polyA sequence. In one preferred embodiment, a vector comprises a polymerase III promoter and a 4-5-thymidine transcription termination signal. The termination signal for polymerase III promoters is typically defined by 5 thymidines, and the transcript is typically cleaved after the second uridine, thereby generating a 3′ UU overhang in the expressed siRNA. The expressed siRNA inserts may be stem-looped RNA inserts. Upon expression, shRNAs are understood to fold into a stem-loop structure. Subsequently, the ends of the shRNAs may be processed to convert the shRNA into siRNA-like molecules. Alternatively, expression vectors may be made that express the sense and antisense strands of siRNAs, and upon expression, these strands anneal in vivo to produce a functional siRNA. Each strand may be expressed from a different vector, or both strands may be expressed from a single vector, according to well-established procedures, as described in Miyagishi, M. (2002) Nature Biotechnol. 20:497-500 and Lee, N. S. et al. (2002) Nature Biotechnol. 20:500-505.

ShRNA sequences may be cloned via a PCR-based strategy. In one embodiment of this strategy, described at www.katahdin.cshl.org:9331/RNAi/docs/Web_version_of_PCR_strategy1.pdf, shRNA sequences are converted into a single approximately 72 nt primer sequence onto which are added 21 nucleotides of homology to the human U6 snRNA promoter. In one embodiment of this procedure, an approximately 29 nucleotide “sense” sequence which ends with a C nucleotide is picked from the coding sequence of the target gene of interest. Second, the actual hairpin is constructed in a 5′ to 3′ orientation with respect to the intended transcript. Third, one or several stem pairings are changed to G-U by altering the sense strand sequence. Finally, the hairpin construct is converted to its “reverse complement” onto which is added approximately 21 nucleotides of homology to the human U6 promoter. All of these steps are automated using the hairpin primer program, “RNAi oligo retriever,” available at www.cshl.org/public/SCIENCE/hannon.html.

PCR is then performed using a plasmid containing the desired promoter as template. In one embodiment, a pGEM1 plasmid (Promega) containing the human U6 locus is used as a template for the PCR reaction. A primer flanking the upstream portion of the U6 or other promoter region and the shRNA primer are used in the PCR amplification reaction under standard conditions. Exemplary PCR conditions include 95° C. for 3 min; 30 cycles of 95° C. for 30 sec, 55° C. for 30 sec, and 72° C. for 1 min; followed by one cycle of 72° C. for 10 min, using Taq polymerase with 4% DMSO and 50 pmoles of each primer. The resulting PCR product may be cloned by any available technique. Such methods include, for example, using the T-A or directional topoisomerase-mediated cloning kit (Invitrogen, Carlsbad, Calif.).

In one embodiment of the invention, expression of the knockout reagent is conditionally regulated. For example, expression may be regulated by a conditional promoter or enhancer, wherein expression of the knockout reagent is regulated by inducing or inhibiting the expression of a regulatory molecule that acts on the conditional promoter or enhancer. Examples of such a system include prokaryotic repressors that can transcriptionally repress a disrupted gene into which an appropriate repressor-binding sequence has been inserted. In certain embodiments, repressors for use in the present invention are sensitive to inactivation by physiologically benign inducing agents. Thus, for example, the lac repressor protein may be used according to the invention to control the expression of a eukaryotic promoter engineered to contain a lacO operator sequence (i.e. regulatable gene expression inhibitor sequence); treatment of the host cell with IPTG will cause the dissociation of the lac repressor from the engineered promoter and allow transcription to occur. Similarly, where the tet repressor is used to control the expression of a eukaryotic promoter which has been engineered to contain a tetO operator sequence, treatment of the host cell with IPTG will cause the dissociation of the tet repressor from the engineered promoter and allow transcription of the disrupted gene.

A variety of conditional expression systems are known and available in the art for use in both cells and animals, and the invention contemplates the use of any such conditional expression system to regulate the expression of a knockdown reagent. In certain embodiments of the invention, the use of prokaryotic repressor or activator proteins is advantageous due to their specificity for a corresponding prokaryotic sequence not normally found in a eukaryotic cell. Specific examples are described supra and include lac and tet-based systems. Other examples of conditional expression systems include hormone-induced gene expression, such as that induced by estrogen or ecdysone or derivates thereof. One example of an ecdysone-based system is the Complete Control® Inducible Mammalian Expression System available from Stratagene (La Jolla Calif.), which utilizes a synthetic ecdysone inducible receptor and a synthetic receptor recognition element that modulates expression of a gene of interest.

It should be understood that the present invention allows for considerable flexibility and a wide range of suitable inducible promoter and corresponding inducing agents, when used. In some embodiments of the invention, the choice of an inducible promoter may be governed by the suitability of the required inducing agent. Factors such as cytotoxicity or indirect effects on nontarget genes may be important to consider. In other instances, the choice may be governed by the properties of the inducible system as a whole. Examples of factors that might be important to consider include the ease with which the system can be introduced into the appropriate cell and the speed and strength with which induction of the system occurs following exposure to an inducing agent. Again, it is reiterated that the particular system chosen to induce or activate an effector of repression through a regulatable gene expression inhibitor sequence may operate in the presence of absence of an inducing agent, depending on the particular system chosen. Thus, in certain embodiments, cells will be maintained in an agent or compound to avoid repression of the disrupted gene, while in other embodiments, an agent or compound will be added to induce repression of a disrupted gene.

Knockdown reagents, including, for example, antisense molecules, ribozymes, double-stranded RNAs and shRNAs, may be designed to target a variety of different regions of a targeted gene or nucleic acid sequence. Generally, target sequences are contained within a transcribed region of a gene or nucleic acid sequence, particularly since many knockdown agents target mRNAs. Target sequences may be located within coding or non-coding regions of a gene or mRNA transcript. In one embodiment of the invention, knockdown reagents are designed to bind and/or target transcribed regions of endogenous genes.

A knockdown molecule for use in the present invention may be designed from database-submitted entries, via data obtained from techniques such as RACE, or via other methods that can determine the identity of the trapped gene, such as through the use of polynucleotide arrays. For instance, one may validate the sequence integrity of identified knockdown molecules, for example, by applying the knockdown molecule to gene arrays and identifying which gene(s) or gene fragment(s) they hybridize to. The individual RNA strands that make up the knockdown molecule can be made recombinantly or synthesized chemically. The resultant knockdown molecule may be introduced into cells of the instant invention by one of any standard techniques such as transfection, lipofection and electroporation, or viral delivery systems, for example, in addition to other methods described above.

2. Ectopic Genes and Conditional Expression Vectors

The invention contemplates the expression of an ectopic gene controlled by a conditionally regulated promoter or enhancer. Systems, promoters, and enhancers for the conditional expression of genes and polynucleotide sequences are well known in the art and examples are described in detail supra. Any conditionally regulated promoter or enhancer and any means of regulating gene expression may be used in practicing the invention.

The ectopic gene that is conditionally expressed is typically a sequence variant of the endogenous gene targeted by the knockdown reagent. Preferably, the ectopic gene is not targeted by a knockdown reagent directed to the corresponding endogenous gene, and the expression of the ectopic gene is not significantly or substantially reduced or affected by a knockdown reagent that targets the corresponding endogenous gene. Accordingly, expression of the ectopic gene in the presence of a knockdown reagent is not reduced by more than 10, 20, 30, 40 or 50% as compared to when not in the presence of a knockdown reagent. Thus, at least one or more sequence changes present in the ectopic variant should reside in the region of the target site for the knockdown reagent directed to the corresponding endogenous gene or polynucleotide sequence. However, the functional polynucleotide or polypeptide expressed from the ectopic variant should possess at least some or all of the functional characteristics associated with the corresponding endogenous gene. For example, if the corresponding endogenous gene expresses a polypeptide with functional properties, then it may be desirable for the ectopic variant to also possess certain or all of these properties. In certain circumstances, the ectopic variant should possess all functional properties of the corresponding endogenous gene, such that expression of the ectopic variant essentially substitutes or compensates for any reduction in expression of the corresponding gene caused by the presence of a knockdown reagent targeted to the endogenous gene.

In some cases, degradation of the endogenous mRNA may lead to the production of a knockdown reagent capable of targeting degradation of mRNAs expressed from the ectopic variant, for example, when a degradation product of the endogenous gene has the identical sequence as a region of the ectopic variant. Without wishing to be bound to any particular theory or mechanism, it is believed that the upon binding of an RNAi reagent to a target sequence, the dsRNA is extended by an RNA-dependent RNA polymerase, thereby creating longer dsRNAs, including sequences corresponding to genomic sequence, which are subsequently degraded and can act as RNAi reagents themselves. Effectively, this amplification reaction, which has been observed in worms, plants and fungi during RNAi or cosuppression, may take place by siRNA-priming of mRNAs and a 5′ to 3′ extension by an RNA-dependent RNA polymerase. These amplified dsRNAs, therefore, should extend to the 5′ end of mRNAs. Several RNA-dependent RNA polymerases involved in this process have been identified, including, for example, Neurospora qde-1, Arabidopsis SDE-1/SGS-2 and C. elegans ego-1. Accordingly, in certain embodiments of the invention, an RNA-dependent RNA polymerase may be introduced into a cell or in vitro reaction, e.g., to facilitate RNAi of other alleles corresponding to a gene-trapped gene. Such polypeptides and polynucleotides may be derived from any species. Examples of such polypeptides and encoding polynucleotide sequences include Dicer (e.g. C. elegans dcr-1), and the C. elegans genes, rde-1 and rde-4, rde-2 and mut-7. Mechanisms of RNA interference are discussed, for example, in Sharp, P. A. and Zamore, P. D. Science 287:2431-2433 (2000) and Sharp, P. A., Genes Dev. 15:485-490 (2001).

To reduce the possibility that a knockdown reagent will target an ectopic variant, as described above, for different alleles of a target gene, ectopic variants may have multiple sequence differences. This may be accomplished by a variety of means, including artificial synthesis of the entire ectopic variant polynucleotide (e.g. RNA or cDNA), including as many substitutions or sequence changes as required to substantially reduce or inhibit the direct and indirect degradation of the ectopic gene's expressed mRNA by a knockdown reagent targeted to the corresponding endogenous gene. In one embodiment, the ectopic gene contains a substituted base at least every 18 nucleotides, and in other embodiments, the ectopic gene contains a substituted base at least every 12, 13, 14, 15, 16, 17, 18, 19, 20, 21, 22, 23, 24, or 25 bases. Thus, in certain embodiments, the ectopic gene contains base substitutions spaced such that they occur at least as frequently as the length of the knockdown reagent targeting the endogenous gene.

Accordingly, a variety of ectopic variants may be useful according to the invention. An ectopic polynucleotide “variant,” as the term is used herein, is a polynucleotide that typically differs from an endogenous gene or polynucleotide sequence in one or more substitutions, deletions, additions and/or insertions. Such variants may be naturally occurring or may be synthetically generated. Moreover, it will be appreciated by those of ordinary skill in the art that, as a result of the degeneracy of the genetic code, there are many variant nucleotide sequences that encode for the same polypeptide as a corresponding endogenous gene. Some of these polynucleotides bear minimal homology to the nucleotide sequence of any native or endogenous gene. Nonetheless, polynucleotides that vary due to differences in codon usage (i.e. degenerate variants) are specifically contemplated by the present invention. In certain embodiments, an ectopic variant is a homolog or ortholog of a different species. In one embodiment, an ectopic variant is a degenerate variant of an endogenous gene. In certain embodiments, the sequence of an ectopic variant may be selected or determined by computer comparison to the sequence of the corresponding endogenous target gene.

In certain embodiments, an ectopic variant will encode a polypeptide containing conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. One skilled in the art would understand that modifications may be made in the structure of the ectopic polynucleotides and encoded polypeptides of the present invention and still obtain a functional molecule that encodes a variant or derivative polypeptide with desirable characteristics. When it is desired to alter the amino acid sequence of a polypeptide to create an equivalent, or even an improved, variant of an endogenous polypeptide, one skilled in the art will typically change one or more of the codons of the encoding DNA sequence.

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties.

Amino acid substitutions may generally be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gin, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes.

In certain embodiments, ectopic polynucleotide variants encompassed by the present invention include those exhibiting at least 70%, 75%, 80%, 85%, 90%, 91%, 92%, 93%, 94%, 95%, 96%, 97%, 98%, or 99% or more identity to an endogenous gene or polynucleotide sequence. One example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides and polypeptides of the invention. Software for performing BLAST analyses is publicly available through the National Center for Biotechnology Information.

In other embodiments, the present invention is directed to ectopic variant polynucleotides that are capable of hybridizing under moderately stringent conditions or highly stringent conditions to a endogenous gene or polynucleotide sequence, or a fragment thereof, or a complementary sequence thereof. Hybridization techniques are well known in the art of molecular biology. For purposes of illustration, suitable moderately stringent conditions for testing the hybridization of a polynucleotide of this invention with other polynucleotides include prewashing in a solution of 5×SSC, 0.5% SDS, 1.0 mM EDTA (pH 8.0); hybridizing at 50° C.-65° C., 5×SSC, overnight; followed by washing twice at 65° C. for 20 minutes with each of 2×, 0.5× and 0.2×SSC containing 0.1% SDS. Suitable highly stringent hybridization conditions include those described above, with the exception that the temperature of hybridization is increased, e.g., to 60-65° C. or 65-70° C.

Ectopic variants may also be selected from the same gene from a different species, naturally occurring mutations that may or may not alter the amino acid sequence encoded by the ectopic variant, or experimentally manipulated changes in the gene that may or may not alter the encoded amino acid sequence.

F. Sequence Tag Targets

Knockdown reagents may also be used to regulate the expression of endogenous or exogenous genes and transcribed polynucleotide sequences by targeting a sequence tag inserted within the transcribed region of an endogenous gene or exogenous polynucleotide sequence. The use of a knockdown reagent that targets a sequence tag present in a transcribed region of a gene or polynucleotide sequence permits the use of a single knockdown reagent to target and reduce expression of a variety of genes. Furthermore, sequence tags that are particularly susceptible to degradation by knockdown reagents may be identified and used to target different genes, thereby facilitating or maximizing the reduction in expression of the targeted gene or transcript.

In the context of regulating the expression of an endogenous gene, any polynucleotide sequence targeted by a knockdown reagent (i.e. a sequence tag) may be inserted into an endogenous gene such that the sequence tag is included in the mRNA transcript expressed from the endogenous gene. A knockdown reagent that targets the sequence tag may then be used to reduce expression of the transcribed mRNA, thereby reducing expression of the allele containing the sequence tag and other alleles of the gene. In the context of an exogenous gene or polynucleotide sequence, a polynucleotide sequence comprising a sequence tag and an exogenous polynucleotide sequence may be introduced into a cell such that an mRNA comprising both the sequence tag and exogenous polynucleotide sequence is expressed. A knockdown reagent that targets the sequence tag may then be used to reduce expression of the introduced polynucleotide sequence. In addition, an exogenous gene may be regulated by first introducing an exogenous sequence into a cell and then introducing a sequence tag into a transcribed region of the exogenous sequence, such that a knockdown reagent that targets the sequence tag reduces expression of the exogenous sequence.

A sequence tag may be any nucleic acid sequence of sufficient length to be specifically recognized by a knockdown reagent. Therefore, the sequence of a sequence tag is preferably not also located within any endogenous. transcribed region of genomic DNA of the cell or organism wherein the knockdown occurs. A sequence tag may be an artificial sequence or it may be a sequence corresponding to a known sequence. A variety of sequences that have been successfully targeted by different knockdown reagents have been identified and are known in the art. Accordingly, any of these known target sequences may be a sequence tag according to the invention. Sequence tags useful in the context of the invention may also be identified by generating different potential tags and corresponding knockdown reagents and testing these combinations for their ability to mediate a reduction in gene expression.

The invention provides methods of reducing the expression of an endogenous gene in a cell, plant or animal by introducing a sequence tag into the endogenous gene and introducing a knockdown reagent that targets the sequence tag into the cell, plant or animal, thereby causing a reduction in expression of the endogenous gene. The sequence tag is typically introduced into a transcribed region of the endogenous gene, and it may be introduced or inserted into translated or untranslated, or coding or non-coding, regions of the gene. For example, a sequence tag may be inserted at the 5′ or 3′ end of the coding region of a gene. A sequence tag may also, for example, be inserted within the 5′ regulatory region or 3′ untranslated region of a gene. In addition, a sequence tag may be inserted either in-frame or not in-frame into a coding region of a gene. Typically, the functional properties and characteristics of the endogenous gene are not affected by the insertion of the sequence tag. Rather, gene function is typically regulated by the introduction or regulation of a knockdown reagent that targets the sequence tag located within the gene. In certain embodiments, the sequence tag may be engineered so that it is expressed as a fusion with the polypeptide encoded by the target gene, and the resulting tagged polypeptide may be identified using an antibody specific for the polypeptide sequence encoded by the sequence tag. Thus, in certain embodiments, the polynucleotide sequence of the sequence tag contains an ATG at the 5′ end.

The invention also provides methods of reducing the expression of an exogenous gene or polynucleotide sequence (e.g. transgene) in a cell, plant, or animal, for example. The exogenous sequence may be stably integrated or transiently present within the cell, plant or animal. For example, the exogenous sequence may be present in an expression vector, including, e.g., plasmid, viral, baculovirus, and episomal vectors. Alternatively, the exogenous sequence may be stably integrated into the genome of a cell, plant, or animal. Typically, the exogenous sequence is introduced in combination with a sequence tag. Thus, a single polynucleotide comprising a sequence tag and an exogenous gene or polynucleotide sequence may be introduced into a cell. The polynucleotide may be an expression vector, a gene trap vector, or a homologous recombination or targeting vector, including those of the invention, for example. Alternatively, an exogenous sequence may be introduced into a cell and a sequence tag may be independently introduced into the cell. The introduction of either or both of the exogenous sequence and the sequence tag into the genome of a cell may be via random insertion or targeted integration into a specific location. Thus, the exogenous sequence and the sequence tag may be introduced into a cell in either temporal order or simultaneously.

The invention also provides a method of regulating the expression of a gene in a cell, plant or animal. The method entails introducing a polynucleotide comprising a sequence tag and an exogenous gene into a cell, such that the gene is expressed in the cell. Thereafter, expression of the gene may be regulated by introducing a knockdown reagent into the cell, such that the knockdown reagent targets the sequence tag and causes a reduction in the expression of the exogenous gene. Transcription of the exogenous gene in the cell may be regulated by either an exogenous promoter or an endogenous promoter. Accordingly, the polynucleotide sequence comprising the sequence tag and exogenous gene further comprises a promoter sequence. In certain embodiments, the promoter driving expression of the exogenous gene is conditionally regulated, by any available method, including those described above. Thus, expression of the exogenous gene may be turned on or off via a conditional promoter and/or the introduction of a knockdown reagent. The knockdown reagent may also or alternatively be expressed via a conditional promoter, thereby providing multiple, regulatable levels of altering expression of the exogenous gene. According to the invention, either or both of the polynucleotide comprising the sequence tag and the exogenous gene and the knockdown reagent may be transiently or stably introduced or expressed within the cell, thereby affording another level of gene regulation.

A variety of exogenous genes may be introduced into a cell, plant or animal and regulated according to a method of the invention. For example, a gene associated with a disease or disorder may be introduced into a cell. Examples of such genes include ras genes, myc genes, and bcl-2 genes. In certain situations, the invention provides a method of replacing an absent, mutated or otherwise dysfunctional gene. One example of such a gene is the p53 gene. In other embodiments, a therapeutic polynucleotide may be introduced into a cell. In addition to providing a missing gene or protein, the therapeutic molecule may act by any of a variety of other means, including, for example, to inhibit the function of another molecule, e.g. a dominant-negative.

In other embodiments of the invention, an exogenous gene may be a reporter or marker gene, such as any of those described previously. Thus, for example, the invention contemplates the insertion of a polynucleotide comprising a sequence tag and a reporter or marker sequence into a gene within a cell, preferably facilitated by a gene trap vector or targeting vector. The disrupted gene containing the sequence tag and reporter or marker sequence expresses a chimeric transcript comprising sequences corresponding to the sequence tag, the marker or reporter, and the disrupted gene. Expression of this transcript may be regulated by an introduced knockdown reagent that targets the sequence tag. Targeting of the sequence tag leads to degradation of the chimeric transcript and the generation of knockdown reagents that target other alleles of the disrupted gene, thereby further reducing expression of the disrupted gene.

In certain embodiments, sequence tags comprise polynucleotide sequences shown to be targets of RNAi attenuation of gene expression in U.S. patent application Ser. No. 20020162126 A1 to Beach et al., which is hereby incorporated by reference in its entirety.

The invention also provides cells comprising sequence tags, with or without knockdown reagents. For example, cells of the invention may comprise a polynucleotide comprising a sequence tag and a gene or polynucleotide sequence and a knockdown reagent that targets the sequence tag. Cells may also comprise a sequence tag and a knockdown reagent that targets the sequence tag. The polynucleotide may or may not also comprise a promoter sequence. Thus, cells may comprise a gene trap vector or targeting vector comprising a sequence tag and a gene, e.g. a reporter or marker gene.

The invention further contemplates libraries, collections, and arrays of cells of the invention. The cells of a library, collection or array may each comprise different disrupted or targeted genes. The libraries, arrays or collections may comprise pools of two or more cells or may comprise individually isolated cells. In addition, the libraries, arrays and collections may comprise multiple groups of vessels.

G. Conditional Expression System

The present invention also provides an efficient method and system for conditionally regulating the expression of a gene of interest. As described above, a variety of conditional expression systems may be used according to the invention to regulate the expression of a gene of interest. For example, the Rev-Tet system and hormonally responsive systems, such as estrogen or ecdysome systems, have been successfully used to conditionally regulate gene expression.

Typically, however, establishing conditional gene expression requires extensive labor and analysis and must be performed individually for each gene of interest. For example, for the Rev-Tet system, two vectors are utilized. One expresses a transcription regulator, while the other comprises a regulated promoter driving expression of a gene of interest. In order to successfully conditionally regulate the gene of interest, a great deal of optimization is required, which involves considerable work for each gene of interest to be conditionally regulated. Variables that effect conditional regulation and require optimization include the level of expression of the transcription regulator and the genomic integration site of the regulated promoter, for example.

To create large numbers of conditionally regulated genes and/or libraries and arrays of conditionally regulated genes according to the procedure generally described above would be tedious and labor-intensive. To reduce or eliminate the necessity of screening and optimizing conditional expression for each gene of interest, the invention provides a method of establishing an optimized conditional expression system using a suitable reporter gene, which can then be used to establish a conditional expression system for any gene of interest.

Essentially, the method of preparing conditional expression systems provided by the invention involves establishing and optimizing any known conditional expression system using a marker or reporter gene. The vector comprising the marker gene also comprises one or more site-specific recombinase sites, which allow a gene of interest to be inserted at these sites. In one embodiment, the vector contains a reporter or marker gene and a single recombinase site 3′ of the marker gene. Thus, a gene of interest may be inserted at the recombinase site using a site-specific recombinase that recognizes the recombinase site, as described supra. The gene of interest may be preceded by an IRES, which is also inserted into the recombinase site and serves to allow expression of the gene of interest from the conditional promoter. Site-specific recombinase sites may be located at the 5′ and 3′ regions of the polynucleotide sequence to be inserted.

In another embodiment, the vector contains a reporter or marker gene with two recombinase sites. In one embodiment, the recombinase sites are situated so as to allow insertion of a gene of interest into the vector, for example, following integration of the vector into the genome. For example, the recombinase sites may be located within or at each end of the multiple cloning site. In another embodiment, the recombinase sites may be located within the vector so as to allow the replacement of marker or reporter sequences with a gene of interest. Thus, in one embodiment, one recombinase site is located 5′ of the marker gene, and one recombinase site is located 3′ of the marker or reporter gene. Thus, the marker or reporter gene may be replaced by a gene of interest using a site-specific recombinase that recognizes the recombinase sites. In certain embodiments, the gene of interest is inserted with a reporter or selectable marker, with or without an IRES. The selectable marker and IRES may be located 5′ of the gene of interest, so that both the selectable marker and gene of interest are expressed from the conditional promoter. Alternatively, the IRES and selectable marker may be 3′ of the gene of interest, thereby allowing expression of both the marker and gene of interest from the conditional promoter. In another embodiment, the selectable marker may be located 3′ of the gene of interest, and another promoter sequence may be located between the gene of interest and the selectable marker, such that expression of the selectable marker is driven from this promoter. This promoter may be any type of promoter, so long as it is capable of driving marker gene expression in a cell of interest. Thus, the promoter may be constitutive, inducible, tissue-specific or temporal-specific. In addition, the promoter may be derived from any source, including, for example, mammalian or viral polynucleotide sequences. The promoter may provide high levels of constitutive marker gene expression, in order to facilitate easy detection of the marker gene product.

In one specific embodiment, the vector comprises recombinase sites flanking a cassette including a marker (e.g. neoR), a multiple cloning site, an IRES (e.g. GTX), and a reporter (e.g. SEAP). The recominbase sites may be those discussed supra, or, alternatively, in certain embodiments, the site-specific recombinase may be derived from lambda phage and recognized by a lambda recombinase. For lambda to integrate into bacterial chromosomes, as it does during lysogenization, it is believed that two proteins catalyze the insertion of the phage DNA into the bacterial chromosome at a specific recombination site (att) present in the genome. The reverse reaction, excision of the phage genome from the E. coli chromosome, is mediated by three proteins—some viral, some bacterial. The presence or absence of a single protein, Xis, and the particular recombination sites involved, control the direction of these recombination reactions. These recombination proteins recognize four types of att recombination sites. In certain embodiments, the B and P types sites are used for integration, and the L and R types for excision. Accordingly, any of these recombination sites may be useful according to the invention. Using these and modified version of these recombination sites, site-specific recombination may be performed both in vivo and in vitro. Methods and reagents for performing site-specific recombination using lambda att sites, for example, are known in the art and commercially available, including, for example, the Gateway Cloning Technology (Invitrogen, Carlsbad, Calif.). Suitable site-specific recombination sites may also be derived from other species, such as, for example, the Streptomyces phage (phi)C31.

In certain embodiments, the vector comprising the regulatable promoter contains a multiple cloning site. The presence of a multiple cloning site facilitates the subcloning or insertion of marker genes or other genes to be regulated. The multiple cloning site may contain one or more restriction enzyme sites. For example, the multiple cloning site may comprise a polynucleotide sequence containing a plurality of restriction enzyme sites, some or all of which may be overlapping in sequence. The presence of multiple restriction enzyme sites may be advantages in that it allows genes to be inserted into the vector using a variety of potential restriction sites. In other embodiments, the vector does not comprise a multiple cloning site. In such embodiments, a marker gene or gene of interest may be inserted into the vector using alternative cloning techniques available in the art, including for example, PCR-based methods.

Any suitable reporter or selectable marker may be used according to the invention, and a variety have been described supra. In certain embodiments, the product of a reporter or selectable marker gene will have enzymatic activity. For example, one preferred marker gene is secretory alkaline phosphatase. In certain embodiments, the produce of the marker or reporter gene will comprise an amino acid sequence capable of specifically binding an antibody. The antibody binding sequence, or epitope, may be a region of the reporter or selectable marker gene product or, alternatively, it may additional sequence engineered to be expressed, for example, as a fusion with the reporter or selectable marker gene product. The antibody binding sequence may be located anywhere throughout the reporter or selectable marker sequence, so long as it is still recognized and bound by its antibody and the reporter or selectable marker retains sufficient activity to allow identification and/or selection of cells expressing the reporter or selectable marker. In certain embodiments, an antibody binding sequence is engineered to be expressed at the amino- or carboxy-terminus of the reporter or selectable marker. Examples of antibody binding sequence that may be used according to the invention include the widely known and used epitope tags, including FLAG, myc, and HA. The presence of the antibody binding site provides another means to identify expression of the product of the reporter or selectable marker gene.

Any gene, polynucleotide sequence, polypeptide, or amino acid sequence of interest may be conditionally expressed according to the invention. In one embodiment, the gene being conditionally expressed encodes a polypeptide sequence. The gene or its gene product may be associated with a particular function, phenotype or disease. Alternatively, the function of the gene may be unknown, and the conditional expression system of the invention may be used to ascertain the function of the gene or gene product and/or its associated phenotype. The conditional expression system may be used to drive expression of, for example, the mRNA of a gene which has been knocked out or whose expression has been knocked down. Accordingly, the invention may be used to restore expression of a knocked-out or knocked-down gene, for example, during certain developmental stages or in certain tissues.

In another embodiment, the conditional expression system of the invention is used to conditionally express a knockdown reagent, such as, for example, an antisense RNA, a ribozyme, or a double-stranded RNAi molecule, such as an siRNA or a shRNA. Thus, the invention allows the conditional knock-down of gene expression, e.g. during certain developmental or temporal stages or in certain cells or tissues. The ability to conditionally knock-out or knock-down gene expression at certain times or for a limited time period will eliminate many of the problems associated with traditional knock-out technologies, both in cells and animals.

The invention, therefore, provides methods, components, and systems to prepare a conditional expression system for any gene. Essentially, the method comprises establishing a conditional expression system using a marker gene and then replacing the marker gene with a gene of interest via site-specific recombination, using molecular and cell biology techniques. Typically, establishing the conditional expression system for the marker gene will utilize two vectors, including a vector expressing a transcription regulator and a vector comprising the marker gene under control of a promoter regulated by the transcriptional regulator. These vectors may be any type of vector available in the art, including, e.g. plasmids, episomes, or viruses. In certain embodiments, retroviral vectors are used. The vectors are introduced into a cell simultaneously or in either order. For example, the vector expressing the transcription regulator may be inserted into cells, and a cell expressing desired levels of the transcription regulator selected to be subsequently inserted with the conditional promoter vector. The vectors may be inserted into the cells by any means available in the art, including, e.g. transfection, electroporation, scrape-loading, or infection. Cells expressing the desired levels of transcription regulator may be identified by a variety of means. For example, expression of the gene product may be directly assayed by western blot or immunoprecipitation using antibodies directed against the transcription regulator or an associated epitope tag. Alternatively, appropriate or desired levels of expression of the transcription regulator may be determined indirectly by assessing the expression of a marker or reporter gene expressed from a promoter regulated by the transcription regulator. This method is particularly useful when both vectors are introduced into a cell at the same time. In certain embodiments, the vectors are stably integrated into the genome of the cell. Levels of expression may be determined both in the presence and absence of the inducer that causes expression of the transcription regulator, in order to identify a cell wherein expression is low or undetectable in the off state, while expression is significantly increased or moderate to high in the on state. Expression of the reporter driven from the conditional promoter is at least two-fold higher in the on state as compared to the off state and may be at least five-fold, at least ten-fold, at least twenty-fold, at least fifty-fold, or greater than fifty-fold higher. The on state is considered the state wherein reporter expression is induced or activated, while the off state is considered the state wherein reporter expression is repressed or deactivated.

After a cell expressing the reporter in a conditionally regulated manner has been identified, a polynucleotide sequence comprising a gene of interest flanked by the same site-specific recombinase sites as those in the conditionally regulated promoter vector is introduced to the cell. The site-specific recombinase that recognizes these sites may also be introduced, either at the same time or prior to or subsequent to the introduction of the gene of interest. Cells are maintained under conditions to permit site-specific recombination, and cells having undergone site-specific recombination are selected. These cells may be selected based on a variety of different criteria, depending on the particular vectors and recombination strategy employed. For example, where the gene of interest has replaced the marker gene, cells no longer expressing the marker gene may be selected or cells expressing the gene of interest in the on state may be selected. Expression of the gene of interest may be determined by a variety of routine techniques, including, for example, RT-PCR or northern blot. Where the gene of interest has been inserted without deleting the marker gene, then expression of the gene of interest may be assayed, as described above. Alternatively, where an additional or different marker or reporter gene has been inserted with the gene of interest, cells may be selected based on their expression of this marker or reporter.

In one embodiment, a plurality of genes may be conditionally regulated according to the invention. Vectors and cells comprising conditionally regulated genes may be pooled or arrayed as described supra. Thus, the effect of conditionally regulating expression (e.g. reducing or increasing expression) of different genes may be determined, and pools or arrays of conditionally regulated genes may be screened to identify a gene associated with a particular function or phenotype.

All of the above U.S. patents, U.S. patent application publications, U.S. patent applications, foreign patents, foreign patent applications and non-patent publications referred to in this specification and/or listed in the Application Data Sheet are incorporated herein by reference, in their entirety. Additional patent applications incorporated by reference include U.S. patent application Ser. No. 10/172,715, U.S. patent application Ser. No. 10/097,431 and U.S. patent application Ser. No. 10/028,970.

The practice of the present invention will employ a variety of conventional techniques of cell biology, molecular biology, microbiology, and recombinant DNA, which are within the skill of the art. Such techniques are fully described in the literature. See, for example, MOLECULAR CLONING: A LABORATORY MANUAL, 2ND ED., ed. by Sambrook, Fritsch, and Maniatis (Cold Spring Harbor Laboratory Press, 1989); and DNA CLONING, VOLUMES I AND II (D. N. Glover ed. 1985).

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A recombinant cell that expresses a gene of interest, comprising a multiplicity of genes that consists of an endogenous gene encoding a first polypeptide and at least one other gene that encodes a second polypeptide having at least 90% sequence identity to said first polypeptide, wherein expression of at least one gene of the multiplicity is significantly inhibited by a knockdown reagent and at least one other gene is not.

2. The recombinant cell of claim 1, wherein the knockdown reagent is an antisense polynucleotide, a ribozyme, or a double-stranded RNA (dsRNA).

3. The recombinant cell of claim 2, wherein the knockdown reagent is a dsRNA.

4. The recombinant cell of claim 3, wherein the dsRNA is encoded by a vector that is expressed in said cell.

5. The recombinant cell of claim 4, wherein the vector comprises a promoter or enhancer operably linked to a polynucleotide encoding said dsRNA.

6. The recombinant cell of claim 5, wherein the promoter comprises a polI, polII or polIII promoter.

7. The recombinant cell of claim 5, wherein the promoter or enhancer are a conditional promoter or enhancer that are acted on by a regulatory molecule.

8. The recombinant cell of claim 7, wherein the regulatory molecule is introduced to said cell.

9. The recombinant cell of claim 7, wherein the regulatory molecule is synthesized by said cell.

10. The recombinant cell of claim 9, wherein the regulatory molecule is encoded by an exogenous gene of said cell.

11. The recombinant cell of claim 9, wherein the regulatory molecule is encoded by an endogenous gene of said cell.

12. The recombinant cell of claim 7, wherein the regulatory molecule increases expression of the knockdown dsRNA in said cell.

13. The recombinant cell of claim 7, wherein the regulatory molecule inhibits expression of the knockdown dsRNA in said cell.

14. The recombinant cell of claim 4, wherein the vector directs synthesis of a RNA molecule that folds into a stem loop structure.

15. The recombinant cell of claim 4, wherein the vector directs synthesis of both the sense and antisense strands of the dsRNA molecule.

16. The recombinant cell of claim 4, wherein the dsRNA targets a translated region of mRNA of one gene of said multiplicity of genes.

17. The recombinant cell of claim 4, wherein the dsRNA targets an untranslated region of mRNA of one gene of said multiplicity of genes.

18. The recombinant cell of claim 1, wherein the gene of interest regulates apoptosis of said cell.

19. The recombinant cell of claim 1, wherein the multiplicity of genes comprises an ectopic gene.

20. The recombinant cell of claim 19, wherein expression of said ectopic gene is under control of a regulatable promoter.

21. The recombinant cell of claim 19, wherein the ectopic gene is a sequence of the endogenous target gene comprising a plurality of base substitutions.

22. The recombinant cell of claim 19, wherein the knockdown reagent inhibits expression of an endogenous gene.

23. The recombinant cell of claim 19, wherein said knockdown reagent inhibits expression of the ectopic gene.

24. The recombinant cell of claim 19, wherein the ectopic gene is integrated into the genome of the host.

25. The recombinant cell of claim 24, whereby the knockdown reagent inhibits expression of the ectopic gene.

26. The recombinant cell of claim 24, wherein said knockdown reagent inhibits expression of the endogenous gene.

27. The recombinant cell of claim 24, wherein expression of the ectopic gene is not reduced by more than 50% by the knockdown reagent.

28. The recombinant cell of claim 27, wherein expression of the ectopic gene is not reduced by more than 20% as compared to when not in the presence of the knockdown reagent.

29. The recombinant cell of claim 28, wherein expression of the ectopic gene is not reduced by more than 10% as compared to when not in the presence of the knockdown reagent.

30. The recombinant cell of claim 29, wherein expression of the ectopic gene is not significantly inhibited as compared to when not in the presence of the knockdown reagent.

31. The recombinant cell of claim 24, wherein the ectopic gene is operably linked to an internal ribosome entry site (IRES).

32. The recombinant cell of claim 31, wherein the ectopic gene is operably linked to a transcription termination sequence.

33. The recombinant cell of claim 31, wherein the ectopic gene is integrated into a chromosome of the cell.

34. The recombinant cell of claim 33, wherein the ectopic gene is integrated by homologous recombination into a specific chromosomal sequence.

35. The recombinant cell of claim 34, wherein the ectopic gene is integrated within the endogenous gene.

36. The recombinant cell of claim 35, wherein the ectopic gene replaces an endogenous gene that is not expressed.

37. The recombinant cell of claim 35, wherein the ectopic gene replaces a dysfunctional endogenous gene.

38. The recombinant cell of claim 35, wherein the ectopic gene replaces a mutant endogenous gene.

39. The recombinant cell of claim 35, wherein the ectopic gene is integrated into one allele of the endogenous gene.

40. The recombinant cell of claim 35, wherein the ectopic gene is integrated into more that one allele of the endogenous gene.

41. The recombinant cell of claim 33, wherein the ectopic gene is integrated distal to the region of the endogenous gene.

42. The recombinant cell of claim 41, wherein the ectopic gene is integrated into an actively transcribed region of the genome.

43. The recombinant cell of claim 33, wherein the ectopic gene is integrated randomly within the genome.

44. The recombinant cell of claim 24, wherein the endogenous gene encodes for a multiplicity of splice variants.

45. The recombinant cell of claim 44, wherein the ectopic gene encodes for one of the multiplicity of splice variants.

46. The recombinant cell of claim 24, wherein expression of the ectopic gene is under control of a regulatable promoter.

47. The recombinant cell of claim 46, wherein the ectopic gene is regulated by an exogenous promoter.

48. The recombinant cell of claim 46, wherein the ectopic gene is regulated by an endogenous promoter.

49. The recombinant cell of claim 24, wherein the ectopic gene modifies transcription of the endogenous gene.

50. The recombinant cell of claim 24, wherein the cell is selected from the group consisting of a stem cell, a primary cell, a cell from a cell line, an immortalized cell and a transformed cell.

51. The recombinant cell of claim 24, wherein the cell is a somatic cell or a germ cell.

52. The recombinant cell of claim 24, wherein the cell is a non-dividing cell or a cell capable of proliferating.

53. The recombinant cell of claim 24, wherein the cell is a normal cell or a diseased cell.

54. The recombinant cell of claim 50, 51 or 52, wherein the cell is a human cell.

55. The recombinant cell of claim 50, 51 or 52, wherein the cell is a stem cell.

56. The recombinant cell of claim 55, wherein the stem cell is an embryonic stem cell.

57. The recombinant cell of claim 50, 51 or 52, wherein the cell is present in an animal.

58. A multiplicity of cells, wherein each cell of the multiplicity of cells is a recombinant cell of claim 24, wherein said multiplicity comprises a first cell that comprises a first ectopic gene, wherein said multiplicity comprises a second cell that comprises a second ectopic gene, and wherein said multiplicity comprises at least one other cell that comprises an ectopic gene not found in any other cell within the multiplicity of cells.

59. The multiplicity of cells of claim 58, wherein said multiplicity of cells consists of an array, wherein said array is comprised at least three members, and wherein each member of said array comprises an ectopic gene not found any other member of the array.

60. The multiplicity of cells of claim 58, wherein said multiplicity of cells consists of an array, wherein said array is comprised at least members, and wherein each member of said array comprises a knockdown reagent not found in any other member of the array.

61. The multiplicity of cells of claims 58, 59 or 60, wherein said multiplicity is useful for screening anti-cancer compounds.

62. A multiplicity of cells for evaluating a candidate anti-cancer compound, comprising: a first recombinant cell of claim 24, wherein the endogenous gene is expressed; and a second recombinant cell of claim 24, wherein the endogenous gene is not expressed.

63. A multiplicity of cells for evaluating a candidate anti-cancer compound, comprising: a first recombinant cell of claim 24, wherein an ectopic gene is expressed; and a second recombinant cell of claim 24, wherein the ectopic gene is not expressed.

64. A method of analyzing a gene of interest in a recombinant cell, comprising:
(a) selecting a cell comprising an endogenous gene that encodes a polypeptide;
(b) integrating into the genome of said cell an ectopic gene comprising a polynucleotide sequence that encodes a polypeptide having at least 90% identity to said polypeptide encoded by said endogenous gene; and
(c) introducing into said cell a knockdown reagent that inhibits expression of either the endogenous gene or the ectopic gene but not both.

65. The method of claim 64, wherein the knockdown reagent is an antisense polynucleotide, a ribozyme, or a double-stranded RNA (dsRNA).

66. The method of claim 65, wherein the knockdown reagent is encoded by a vector.

67. The method of claim 65, wherein the knockdown reagent is a dsRNA.

68. The method of claim 67, wherein the dsRNA is encoded by a vector that is expressed in the recombinant cell.

69. The method of claim 68, wherein the vector encoding the dsRNA comprises a promoter or enhancer that functions in the recombinant cell.

70. The method of claim 69, wherein the promoter comprises a poll, polII or polIII promoter.

71. The method of claim 69, wherein the promoter or enhancer are a conditional promoter or enhancer that are acted on by a regulatory molecule.

72. The method of claim 71, wherein the regulatory molecule is introduced to the cell.

73. The method of claim 71, wherein the regulatory molecule is synthesized by the cell.

74. The method of claim 73, wherein the synthesis of the regulatory molecule is directed by an exogenous gene.

75. The method of claim 73, wherein the synthesis of the regulatory molecule is directed by an endogenous gene.

76. The method of claim 71, wherein the regulatory molecule increases expression of the knockdown dsRNA by the cell.

77. The method of claim 71, wherein the regulatory molecule inhibits expression of the knockdown dsRNA by the cell.

78. The method of claim 68, wherein the vector directs synthesis of a RNA molecule that folds into a stem loop structure.

79. The method of claim 68, wherein the vector directs synthesis of both the sense and antisense strands of the dsRNA molecule.

80. The method of claim 67, wherein the dsRNA interacts with a translated region of mRNA encoded by the endogenous target gene.

81. The method of claim 67, wherein the dsRNA interacts with an untranslated region of mRNA encoded by the endogenous target gene.

82. The method of claim 64, wherein the gene of interest regulates apoptosis of a cancer cell.

83. The method of claim 64, wherein the knockdown reagent inhibits expression of the ectopic gene.

84. The method of claim 64, wherein the knockdown reagent inhibits expression of the endogenous gene.

85. The method of claim 84, wherein the polynucleotide sequence of the ectopic gene comprises a plurality of base substitutions in the polynucleotide sequence of the endogenous gene.

86. The method of claim 85, wherein the polynucleotide sequence of the ectopic gene encodes a polypeptide that is identical to that encoded by the endogenous gene.

87. The method of claim 64, wherein treating the recombinant cells to a candidate anti-cancer compound to examine the function of the gene of interest.

88. The method of claim 64, wherein the ectopic gene further comprises a sequence tag that is present in a transcribed region of the gene.

89. The method of claim 88, wherein the knockdown reagent targets the sequence tag within the ectopic gene.

90. The method of claim 64, further comprising introducing a homologous recombination vector capable of inserting a sequence tag within a transcribed region of the endogenous gene.

91. The method of claim 90, wherein the knockdown reagent targets the sequence tag within the endogenous gene.

92. The method of claim 64, wherein the polypeptide encoded by the ectopic gene is functionally identical to that encoded by the endogenous gene.

93. The method of claim 64, wherein the endogenous gene consists of a multiplicity of splice variants and wherein the ectopic gene encodes for one of the multiplicity of splice variants.

94. The method of claim 93, wherein the endogenous gene consists of a multiplicity of sequence variants and wherein the ectopic gene encodes for one of the multiplicity of sequence variants.

95. The method of claim 64, wherein the endogenous gene consists of a multiplicity of mutants and wherein the ectopic gene encodes for one of the multiplicity of mutants.

96. The method of claim 64, wherein the endogenous gene is associated with anchorage independent growth, production of angiogenic factor, growth factor independence, growth in low nutrients, autocrine growth, alteration of activation of signal transduction pathways, tumorigenesis, metastasis, or cell cycle profiles.

97. The method of claim 96, wherein the signal transduction pathways include Ras, p53, growth factor receptor signaling, and lipid metabolism.

98. The method of claim 64, wherein a targeting vector comprising the ectopic gene is introduced into the cell and integrates into the cellular genome.

99. The method of claim 98, wherein the targeting vector integrates into the cellular genome by homologous recombination.

100. The method of claim 99, wherein the targeting vector comprises the ectopic gene operably linked to an internal ribosome entry site (IRES).

101. The method of claim 100, wherein the targeting vector comprises the ectopic gene operably linked to a transcription termination sequence.

102. The method of claim 101, wherein the ectopic gene is integrated into a chromosome of the cell.

103. The method of claim 102, wherein the ectopic gene is integrated by homologous recombination into a specific chromosomal sequence.

104. The method of claim 103, wherein the ectopic gene is integrated within the endogenous gene.

105. The method of claim 103, wherein the ectopic gene is integrated distal to the region of the endogenous gene.

106. The method of claim 105, wherein the ectopic gene is integrated into an actively transcribed region of the genome.

107. The method of claim 102, wherein the ectopic gene is integrated randomly within the genome.

108. The method of claim 102, further comprising the steps of integrating into the genome of the cell a second ectopic gene comprising a polynucleotide sequence that encodes a polypeptide having at least 90% identity to said polypeptide encoded by said endogenous gene; and introducing into said cell a knockdown reagent that inhibits expression of either the endogenous gene or one or both of the ectopic genes.

109. The method of claim 102, wherein the expression of the ectopic gene is inhibited by the knockdown reagent.

110. The method of claim 102, wherein the expression of the endogenous gene is inhibited by the knockdown reagent.

111. The method claim 102, comprising the additional step of treating the recombinant cells to candidate anti-cancer compounds.

112. The method claim 111, wherein the candidate anti-cancer compounds are selected from the group consisting of small molecules, peptides, polypeptides and nucleic acids.

113. The method claim 64, comprising the additional step of treating the recombinant cells to candidate compounds that inhibit the expression or activity of a product of the gene of interest.

114. The method claim 64, comprising the additional step of treating the recombinant cells to candidate compounds that compensates for the loss of expression of the gene of interest.

115. An expression system for expressing a gene of interest in a target cell, comprising:
(a) an ectopic gene that encodes a polypeptide having at least 90% identity to a polypeptide encoded by an endogenous gene in said cell,
(b) a knockdown reagent that inhibits expression of one gene selected from the group comprising the endogenous gene and the ectopic gene.

116. The expression system of claim 115, wherein the knockdown reagent is an antisense polynucleotide, a ribozyme, or a double-stranded RNA (dsRNA).

117. The expression system of claim 116, wherein the knockdown reagent is encoded by a vector.

118. The expression system of claim 116, wherein the knockdown reagent is a dsRNA.

119. The expression system of claim 118, wherein the dsRNA is short interfering RNA (siRNA) or short hairpin RNA (shRNA).

120. The expression system of claim 118, wherein the dsRNA is prepared by in vitro transcription.

121. The expression system of claim 118, wherein the dsRNA is prepared by chemical synthesis.

122. The expression system of claim 118, wherein the dsRNA is encoded by a vector that is capable of being expressed in a mammalian cell.

123. The expression system of claim 122, wherein the vector encoding the dsRNA comprises a promoter or enhancer that function in a mammalian cell.

124. The expression system of claim 123, wherein the promoter comprises a polI, polII or polIII promoter.

125. The expression system of claim 123, wherein the promoter or enhancer are a conditional promoter or enhancer that are acted on by a regulatory molecule.

126. The expression system of claim 122, wherein the vector encodes a RNA molecule that folds into a stem loop structure.

127. The expression system of claim 122, wherein the vector encodes both the sense and antisense strands of the dsRNA molecule.

128. The expression system of claim 122, wherein the dsRNA has homology to a translated region of mRNA encoded by the endogenous target gene.

129. The expression system of claim 122, wherein the dsRNA has homology to an untranslated region of mRNA encoded by the endogenous target gene.

130. The expression system of claim 115, wherein the gene of interest regulates apoptosis of a cancer cell.

131. The expression system of claim 115, wherein the ectopic gene is a sequence of the endogenous target gene comprising a plurality of base substitutions.

132. The expression system of claim 131, wherein the ectopic gene is a degenerate variant of the sequence of the endogenous target gene.

133. The expression system of claim 115, wherein the ectopic gene further comprises a sequence tag that is present in a transcribed region of the gene.

134. The expression system of claim 133, wherein the knockdown reagent targets the sequence tag within the ectopic gene.

135. The expression system of claim 115, further comprising a homologous recombination vector for inserting a sequence tag within a transcribed region of the endogenous gene.

136. The expression system of claim 135, wherein the endogenous gene of the target cell further comprises a sequence tag that is present in a transcribed region of the gene.

137. The expression system of claim 115, wherein the polypeptide encoded by the ectopic gene is functionally identical to that encoded by the endogenous gene.

138. The expression system of claim 115, wherein expression of said ectopic gene is under control of a regulatable promoter.

139. The expression system of claim 115, wherein the ectopic gene is a sequence of the endogenous target gene comprising a plurality of base substitutions.

140. The expression system of claim 115, wherein the knockdown reagent inhibits expression of the endogenous gene.

141. The expression system of claim 115, wherein said knockdown reagent inhibits expression of the ectopic gene.

142. The expression system of claim 115, wherein expression of the ectopic gene is not reduced by more than 50% as compared to when not in the presence of the knockdown reagent.

143. The expression system of claim 142, wherein expression of the ectopic gene is not reduced by more than 20% as compared to when not in the presence of the knockdown reagent.

144. The expression system of claim 143, wherein expression of the ectopic gene is not reduced by more than 10% as compared to when not in the presence of the knockdown reagent.

145. The expression system of claim 144, wherein expression of the ectopic gene is not significantly inhibited by the knockdown reagent.

146. The expression system of claim 115, wherein the ectopic gene is integrated into the genome of the host.

147. The expression system of claim 146, wherein the ectopic gene is operably linked to an internal ribosome entry site (IRES).

148. The expression system of claim 147, wherein the ectopic gene is operably linked to a transcription termination sequence.

149. The expression system of claim 148, wherein the ectopic gene is integrated into a chromosome of the cell.

150. The expression system of claim 149, wherein the ectopic gene is integrated by homologous recombination into a specific chromosomal sequence.

151. The expression system of claim 150, wherein the ectopic gene is integrated within the endogenous gene.

152. The expression system of claim 151, wherein the ectopic gene is integrated into one allele of the endogenous gene.

153. The expression system of claim 151, wherein the ectopic gene is integrated into more that one allele of the endogenous gene.

154. The expression system of claim 151, wherein the endogenous gene encodes for a multiplicity of splice variants.

155. The expression system of claim 154, wherein the ectopic gene encodes for one of the multiplicity of splice variants.

156. The expression system of claim 150, wherein the ectopic gene is integrated distal to the region of the endogenous gene.

157. The expression system of claim 156, wherein the ectopic gene is integrated into an actively transcribed region of the genome.

158. The expression system of claim 146, wherein the ectopic gene is regulated by an exogenous promoter.

159. The expression system of claim 146, wherein the ectopic gene is regulated by an endogenous promoter.

160. The expression system of claim 146, wherein the ectopic gene modifies transcription of the endogenous gene.

161. The expression system of claim 146, wherein the ectopic gene is integrated randomly within the genome.

162. The expression system of claim 146, wherein the ectopic gene replaces an endogenous gene that is not expressed.

163. The expression system of claim 146, wherein the ectopic gene replaces a dysfunctional endogenous gene.

164. The expression system of claim 146, wherein the ectopic gene replaces a mutant endogenous gene.

165. The expression system of claim 146, wherein the cell is selected from the group consisting of a stem cell, a primary cell, a cell from a cell line, an immortalized cell and a transformed cell.

166. The expression system of claim 146, wherein the cell is a somatic cell or a germ cell.

167. The expression system of claim 146, wherein the cell is a non-dividing cell or a cell capable of proliferating.

168. The expression system of claim 146, wherein the cell is a normal cell or a diseased cell.

169. The expression system of claim 165, 166 or 167, wherein the cell is a human cell.

170. The expression system of claim 165, 166 or 167, wherein the cell is a stem cell.

171. The expression system of claim 170, wherein the stem cell is an embryonic stem cell.

172. The expression system of claim 165, 166 or 167, wherein the cell is present in an animal.
